(19) United States
(12) Patent Application Publication
(10) Pub. No.: US 2003/0049647 A1
(43) Pub. Date: Mar. 13, 2003

(54) Use of nucleic acid libraries to create toxicological profiles

(76) Inventors: Bassil Dahiyat, Atladena, CA (US); Min Li, Lutherville, MD (US)

Correspondence Address: FISH & NEAVE, 1251 AVENUE OF THE AMERICAS, 50TH FLOOR, NEW YORK, NY, 10020-1105, US

(21) Appl. No.: 10/082,671

(22) Filed: Feb. 22, 2002

Related U.S. Application Data
(60) Provisional application No. 60/270,781, filed on Feb. 22, 2001.

(51) Int. Cl. C12Q 1/68; G01N 33/53; G01N 33/543

(52) U.S. Cl. 435/6; 435/7.1; 436/518

(57) ABSTRACT

This invention features methods of generating toxicity profiles of compounds by screening expression libraries with the compounds. The profiles contain lists of proteins (or other biomolecules) to which the compounds bind and optionally information on the strength of binding.

### CROSS REFERENCE TO RELATED APPLICATION

Under 35 U.S.C. § 119(e)(1), this application claims the benefit of prior U.S. Provisional Application No. 60/270,781, filed Feb. 22, 2001.

### FIELD OF THE INVENTION

This invention relates to the use of nucleic acid libraries encoding nucleic acid modifying enzyme fusion proteins in establishing toxicological profiles of given compounds.

### BACKGROUND OF THE INVENTION

The availability of genome-wide DNA sequence information and the development of high throughput DNA array technology allow for large scale monitoring of the changes in gene expression pattern in response to various external stimuli, e.g., drugs or environmental pollutants. See, e.g., Nuwaysir et al., Mol Carcinog 24(3):153-9 (1999); Haborkorn, Eur. J Nucl Med 29(1):115-32 (2002); Altman et al., Annu Rev Pharmacol Toxicol 42:113-33 (2002); Mancinelli et al., AAPS PharmSci 2(1):E4 (2002); Los et al., Cytometry 47(1):66-71 (2002); Bartosiewicz, Arch Biochem Biophys 376(1):66-73 (2000); Steiner, Ann N Y Acad Sci 919:48-51 (2000); and Ruepp et al., Toxicol Sci 65(1):135-50 (2002).

Using this technology in toxicological analyses, one can predict the toxicity of a candidate pharmaceutical compound before going through a clinical trial. This approach has been termed toxicogenomics. More specifically, by monitoring changes in gene expression pattern in response to a chemical compound, one can correlate the changes with the toxicity level of a compound. Genes monitored for this purpose usually include, for example: xenobiotic metabolizing enzymes (e.g., various isoforms of p450); proteins associated with glutathione regulation, DNA repair, transcription regulation, structural maintenance, cell cycle control, signal transduction, and/or apoptosis; heat shock proteins; and housekeeping genes. See, e.g., Nuwaysir et al., supra; Burchiel et al., Toxicology Sciences 59:193-195 (2001). Toxicogenomic information can be combined with other classical toxicological test results to provide a comprehensive profile for a given chemical's toxicity “potential.”

### SUMMARY OF THE INVENTION

This invention provides new methods for determining, cataloging and predicting the interactions between given chemical compounds and biomolecules (e.g., proteins and peptides) in vivo. In these methods, a given chemical compound is used to screen an expression library to identify proteins that interact with the compound. By knowing what proteins that the compound can bind to, one can create a toxicological profile of this compound that is indicative of the toxicity of the compound in vivo. Such profiling information will improve the risk assessment process in drug development as well as in setting environmental and occupational health standards.

Accordingly, this invention features a method of creating a protein (including peptide)-binding profile of a test compound. This method includes the following steps. First, the test compound is contacted with a library (i.e., a plurality) of nucleic acid-protein (NAP) conjugates, wherein each of said NAP conjugates comprises (a) a fusion protein comprising (i) a nucleic acid modifying (NAM) enzyme (e.g., a Rep protein) and (ii) a candidate protein; and (b) an expression vector comprising (i) a fusion nucleic acid comprising a coding sequence for said NAM enzyme and a coding sequence for the candidate protein, and (ii) an enzyme attachment sequence (EAS); wherein the EAS and the NAM enzyme are covalently linked, and wherein at least two of the NAP conjugates in the library contain different candidate proteins. Then, one determines whether the test compound binds to any of the NAP conjugates. If so, one can then identify the candidate protein to which the test compound binds by determining the nucleotide composition of the candidate protein's coding sequence, which is part of the expression vector linked covalently to this candidate protein. This method allows for the creation of a protein-binding profile of the test compound — a profile that includes information on the identities of the proteins to which the test compound binds, and if binding affinity is determined concurrently or in an additional step, also the information on the strength of the binding (binding affinity as indicated by, e.g., the dissociation constant). These profiles can then be used to evaluate and predict toxicity and other biological activities of the test compounds. In general, compounds having substantial similar profiles may cause identical or similar biological effects in vivo.

The invention features also a method of determining the toxicity of a compound. In this method, the protein-binding profile of a compound having a known toxic effect in an animal species (e.g., mice, rats, primates, or humans) is compared to the protein-binding profile of a test compound. Both profiles are obtained by screening an expression library with the candidate proteins' coding sequences derived from this animal species. Substantial similarity between the two profiles will suggest that the test compound has that toxic effect (albeit possibly to a different degree) in this animal species as well.

Another method embraced within the invention involves comparing two or more protein-binding profiles of a test compound obtained with libraries derived from different animal species, where the toxicity (or lack of it) of the compound has been tested or otherwise known in one of the species. Substantial similarity between the profiles will suggest that the test compound is toxic (or not toxic) in the untested animal species as well.

Another method embraced within the invention involves comparing the protein-binding profiles of a test compound obtained with libraries derived from different organs of the same animal species. The toxicity (or lack of it) of the test compound has been tested or otherwise known in one of the organs. Substantial similarity between the profiles will suggest that the test compound is toxic (or not toxic) in the untested organ as well.

Yet another method of this invention involves comparing the protein-binding profiles of a test compound obtained with libraries derived from an animal species at different developmental stages (e.g., different ages or time points of embryonic development, different genders, or otherwise different physiological conditions). The toxicity (or lack of it) of the test compound has been tested or otherwise known in this animal species at one of the developmental stages. Substantial similarity between the profiles will suggest that the test compound is toxic (or not toxic) in the untested developmental stages as well.

Also included in this invention is a method of determining the toxicity of a compound, involving determining whether the protein-binding profile of the test compound includes one or more one or more of the following: liver enzymes; cytochrome proteins; proteins encoded by multiple drug resistance genes; p450 (including the different isoforms); and proteins associated with glutathione regulation, DNA repair, transcription regulation, structural maintenance, cell cycle control, and/or apoptosis; heat shock proteins; and housekeeping genes. Inclusion of one or more of such proteins in the profile will suggest that the compound is toxic in this animal species.

In these methods, the contacting step can be performed in a cell free system, or inside a cell. In one embodiment, the NAP conjugates are produced by a eukaryotic host cell (e.g., a mammalian, insect or yeast cell) containing the expression vectors; and the contacting step can occur prior to or after lysing the eukaryotic host cels. In some embodiments, the NAM enzyme is a Rep protein. In some embodiments, the coding sequences for candidate proteins are derived from a cDNA library. Some of the methods of this invention further comprise the step of determining the binding affinity between the test compound and the bound NAP conjugate.

As used herein, toxicity is defined as disturbance of normal cellular function or structure. A toxic effect can be indicated, for example, by a compound's ability to affect cellular functions in an undesired manner, such as causing undesired apoptosis, cell growth, dedifferentiation, inability of the affected cell to interact with other cells (e.g., adhesion to other cells, secretion of bioactive substances or otherwise transmitting signals to activate or inactivate other cells, etc.) in a normal way. Two different compounds may be considered as having the same toxic effect even though the degrees of the effect are different.

As used herein, coding sequences “derived from” an animal species refer to coding sequences that are isolated from a tissue source of this species. However, sequence changes (substitutions, deletions, and/or insertions) are permitted, for the purposes of, for example, facilitating cloning, isolation, detection or other desired manipulation.

“Substantial similarity” between two protein-binding profiles means that the second profile contains at least about 60% (e.g., at least about 70%, 80%, 90%, or 95%) of the protein species contained in the first profile.

This invention provides several advantages over other high throughput toxicity profiling methods, such as the toxicogenomic and proteomic (infra) methods. Those methods do not provide information concerning the protein targets to which a test compound bind. They merely monitor genes or gene products whose expression changes in response to the test chemical. In many cases, such changes correlate with, but are not causal to, the effect of the test compound. In contrast, the new method provides information on the direct cellular targets of a test compound.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. The materials, methods, and examples are illustrative only and not intended to be limiting. Throughout this specification and claims, the word “comprise,” or variations such as “comprises” or “comprising,” will be understood to imply the inclusion of a stated integer or group of integers but not the exclusion of any other integer or group of integers.

Other features and advantages of the invention will be apparent from the following detailed description, and from the claims.

### DETAILED DESCRIPTION OF THE DRAWINGS

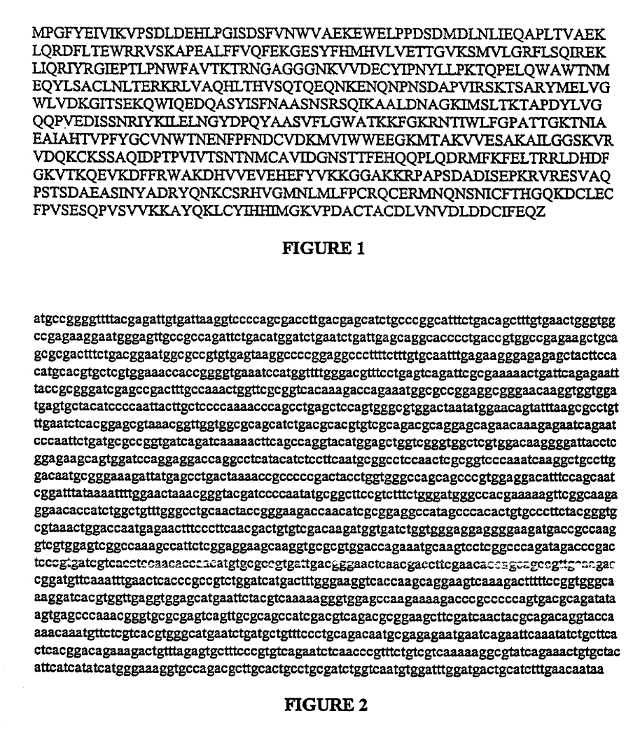

FIG. 1 depicts the nucleotide sequence (SEQ ID NO:55) of Rep78 isolated from adeno-associated virus 2.

FIG. 2 depicts the amino acid sequence (SEQ ID NO:8) of Rep78 isolated from adeno-associated virus 2.

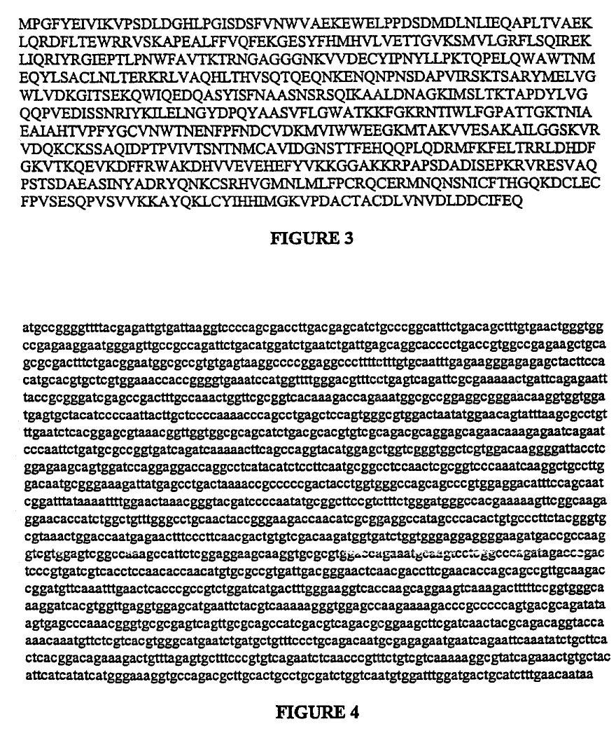

FIG. 3 depicts the nucleotide sequence (SEQ ID NO:9) of major coat protein A isolated from adeno-associated virus 2.

FIG. 4 depicts the amino acid sequence (SEQ ID NO:10) of major coat protein A isolated from adeno-associated virus 2.

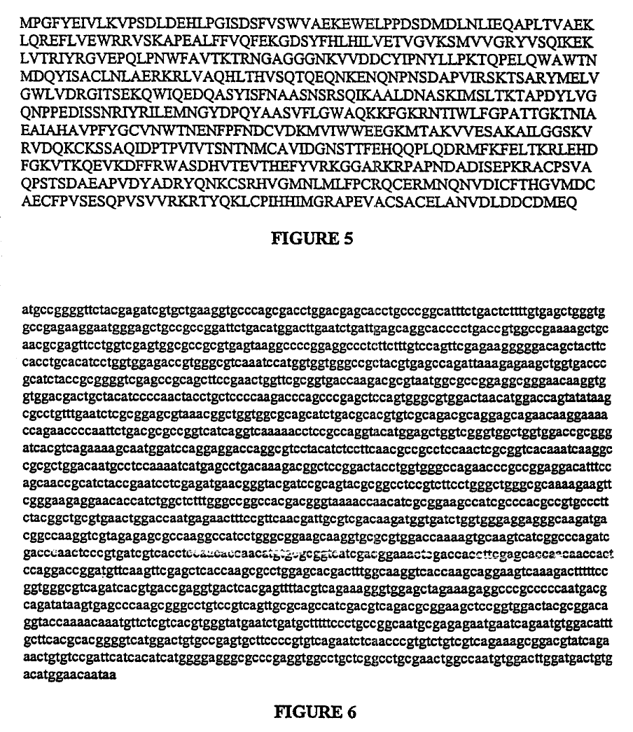

FIG. 5 depicts the nucleotide sequence (SEQ ID NO:11) of a Rep protein isolated from adeno-associated virus 4.

FIG. 6 depicts the amino acid sequence (SEQ ID NO:12) of a Rep protein isolated from adeno-associated virus 4.

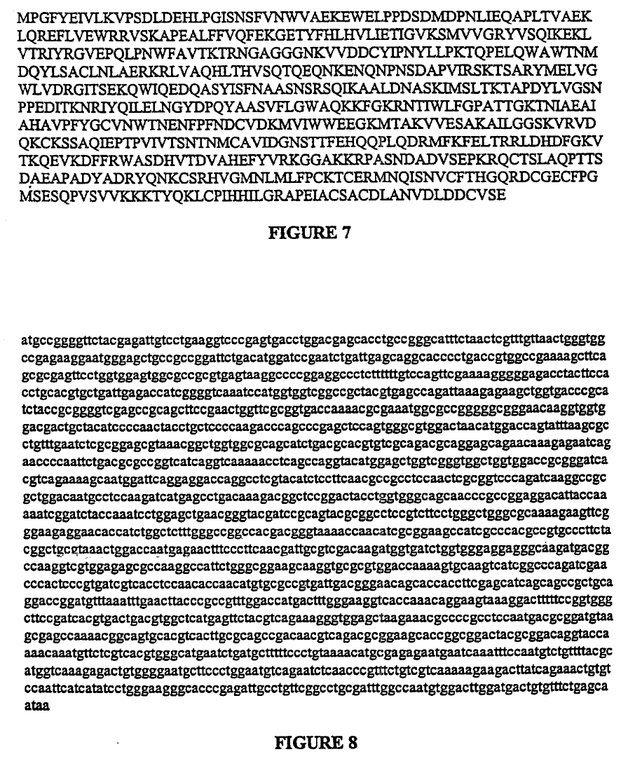

FIG. 7 depicts the nucleotide sequence (SEQ ID NO:13) of Rep78 isolated from adeno-associated virus 3B.

FIG. 8 depicts the amino acid sequence (SEQ ID NO:14) of Rep78 isolated from adeno-associated virus 3B.

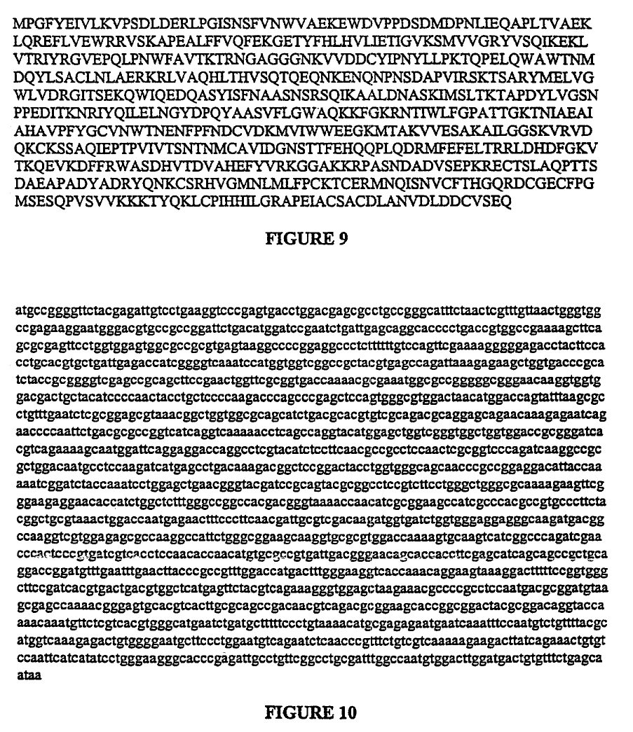

FIG. 9 depicts the nucleotide sequence (SEQ ID NO:15) of a nonstructural protein isolated from adeno-associated virus 3.

FIG. 10 depicts the amino acid sequence (SEQ ID NO:16) of a nonstructural protein isolated from adeno-associated virus 3.

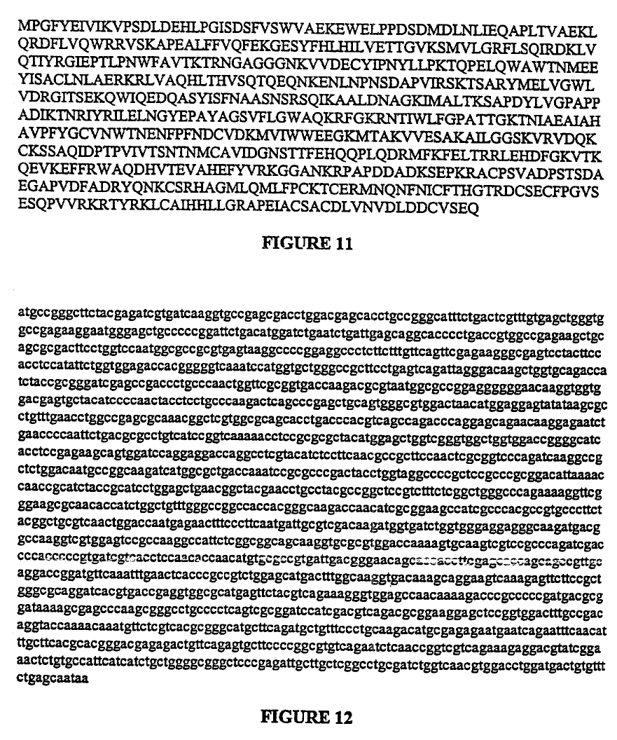

FIG. 11 depicts the nucleotide sequence (SEQ ID NO:17) of a nonstructural protein isolated from adeno-associated virus 1.

FIG. 12 depicts the amino acid sequence (SEQ ID NO:18) of a nonstructural protein isolated from adeno-associated virus 1.

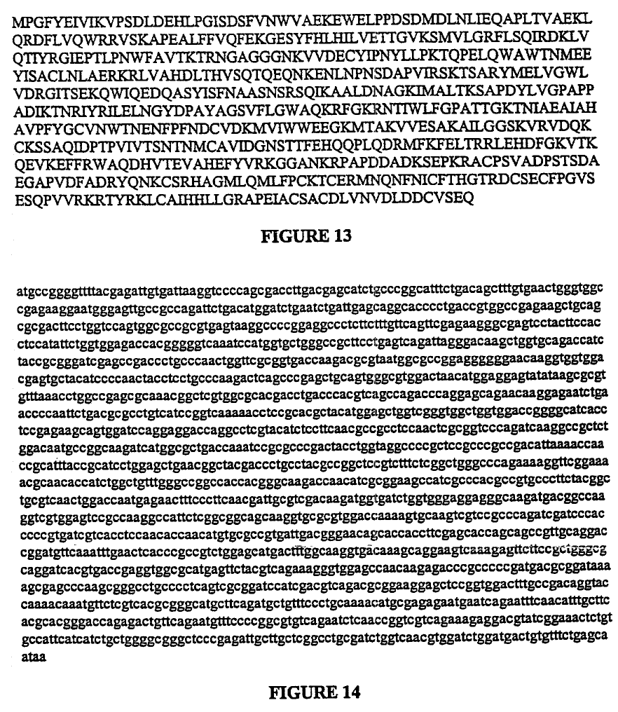

FIG. 13 depicts the nucleotide sequence (SEQ ID NO:19) of Rep78 isolated from adeno-associated virus 6.

FIG. 14 depicts the amino acid sequence (SEQ ID NO:20) of Rep78 isolated from adeno-associated virus 6.

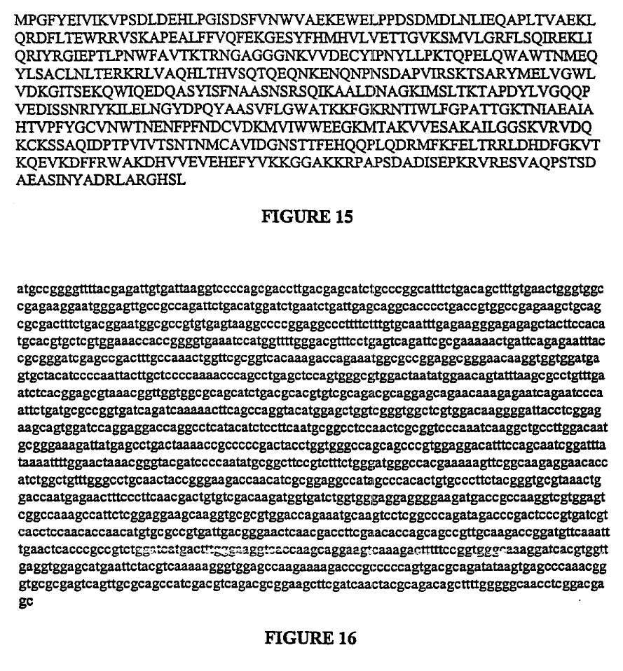

FIG. 15 depicts the nucleotide sequence (SEQ ID NO:21) of Rep68 isolated from adeno-associated virus 2.

FIG. 16 depicts the amino acid sequence (SEQ ID NO:22) of Rep68 isolated from adeno-associated virus 2.

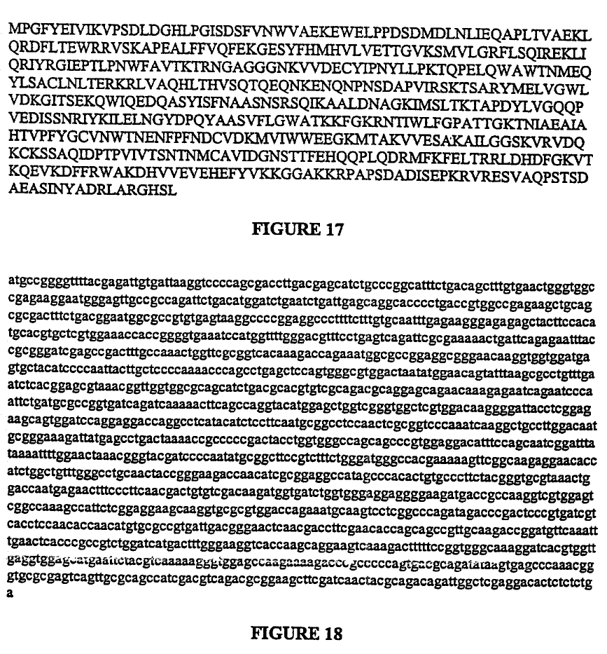

FIG. 17 depicts the nucleotide sequence (SEQ ID NO:23) of major coat protein A′ (alt.) isolated from adeno-associated virus 2.

FIG. 18 depicts the amino acid sequence (SEQ ID NO:24) of major coat protein A′ (alt.) isolated from adeno-associated virus 2.

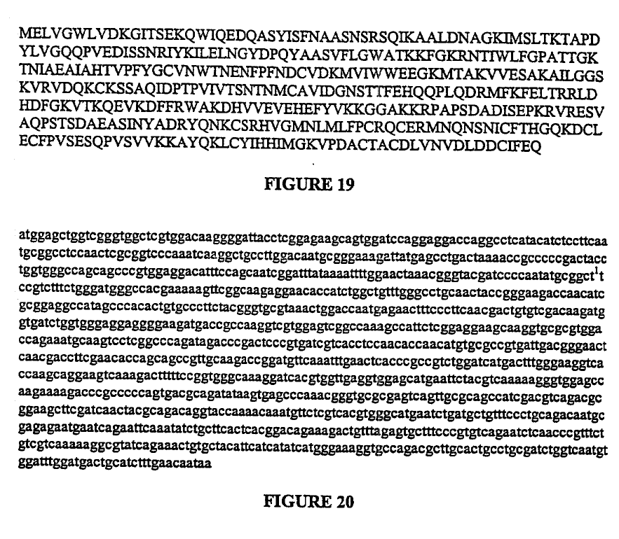

FIG. 19 depicts the nucleotide sequence (SEQ ID NO:25) of major coat protein A″ (alt.) isolated from adeno-associated virus 2.

FIG. 20 depicts the amino acid sequence (SEQ ID NO:26) of major coat protein A″ (alt.) isolated from adeno-associated virus 2.

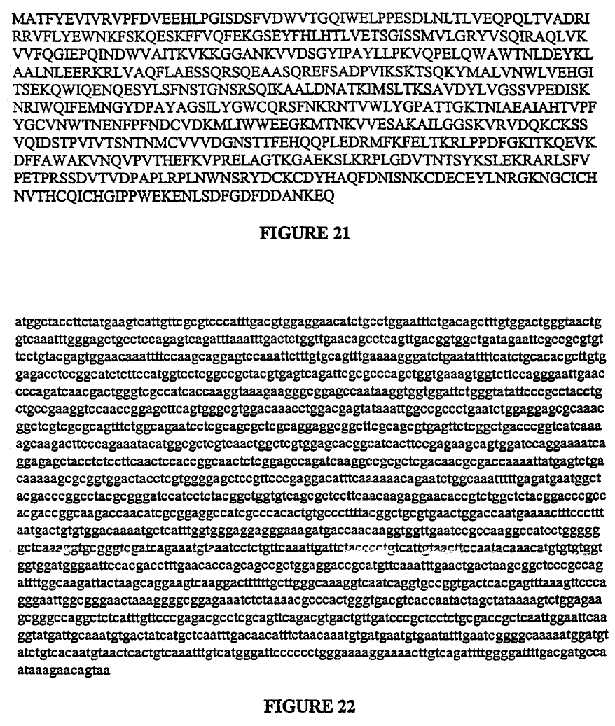

FIG. 21 depicts the nucleotide sequence (SEQ ID NO:27) of a Rep protein isolated from adeno-associated virus 5.

FIG. 22 depicts the amino acid sequence (SEQ ID NO:28) of a Rep protein isolated from adeno-associated virus 5.

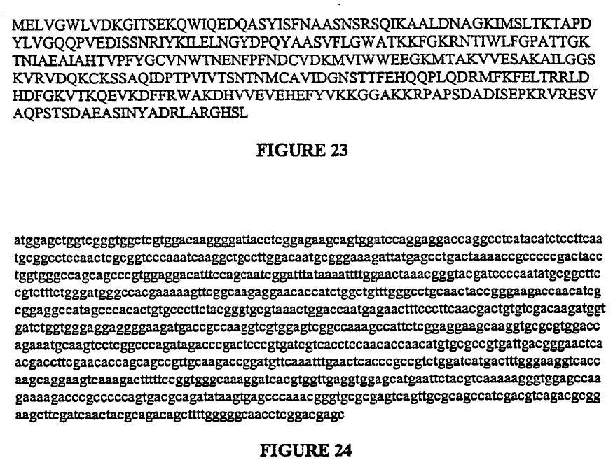

FIG. 23 depicts the nucleotide sequence (SEQ ID NO:29) of major coat protein Aa (alt.) isolated from adeno-associated virus 2.

FIG. 24 depicts the amino acid sequence (SEQ ID NO:30) of major coat protein Aa (alt.) isolated from adeno-associated virus 2.

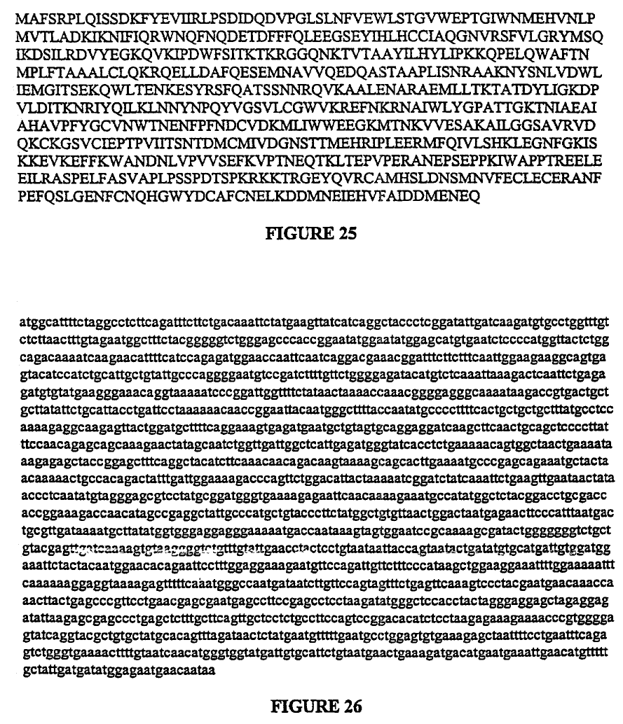

FIG. 25 depicts the nucleotide sequence (SEQ ID NO:31) of a Rep protein isolated from Barbarie duck parvovirus.

FIG. 26 depicts the amino acid sequence (SEQ ID NO:32) of a Rep protein isolated from Barbarie duck parvovirus.

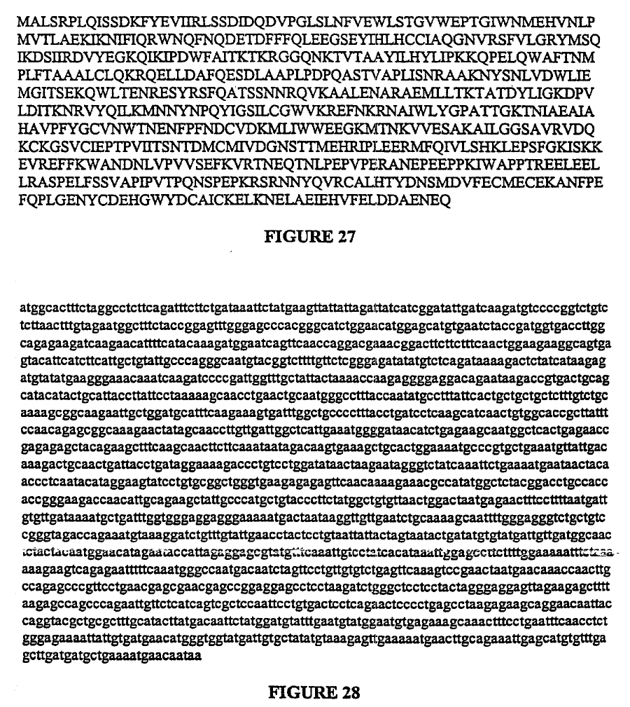

FIG. 27 depicts the nucleotide sequence (SEQ ID NO:33) of a Rep protein isolated from goose parvovirus.

FIG. 28 depicts the amino acid sequence (SEQ ID NO:34) of a Rep protein isolated from goose parvovirus.

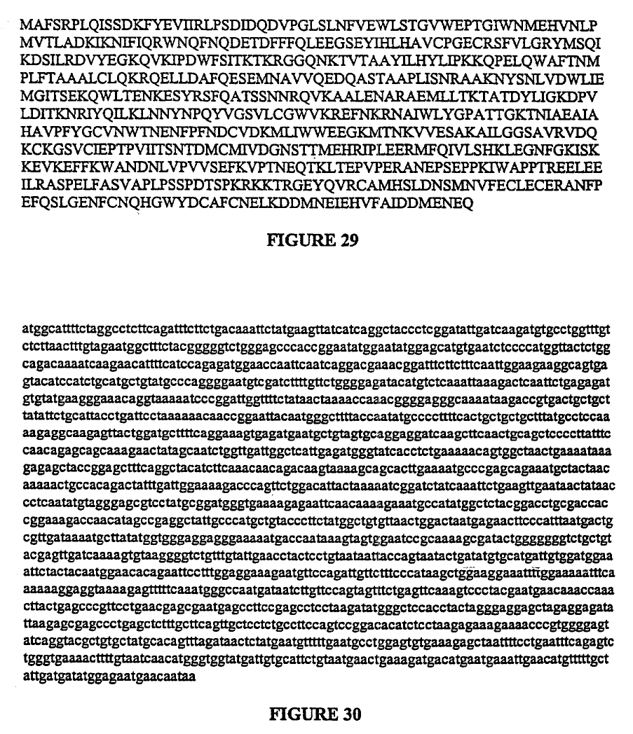

FIG. 29 depicts the nucleotide sequence (SEQ ID NO:35) of NS1 isolated from muscovy duck parvovirus.

FIG. 30 depicts the amino acid sequence (SEQ ID NO:36) of NS1 isolated from muscovy duck parvovirus.

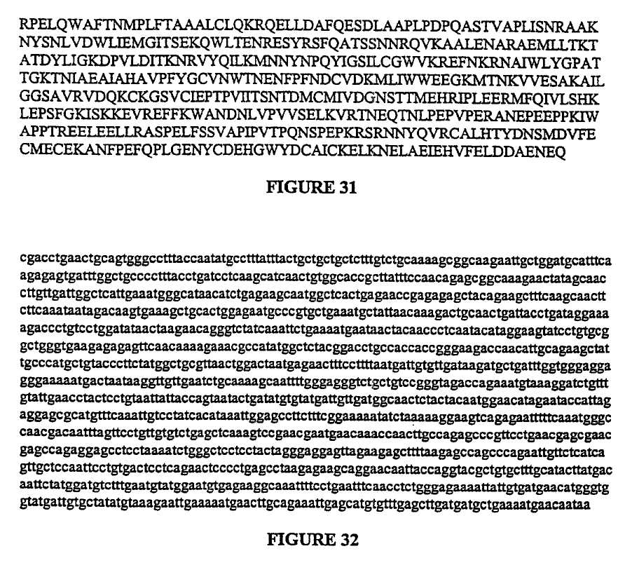

FIG. 31 depicts the nucleotide sequence (SEQ ID NO:37) of NS1 isolated from goose parvovirus.

FIG. 32 depicts the amino acid sequence (SEQ ID NO:38) of NS1 isolated from goose parvovirus.

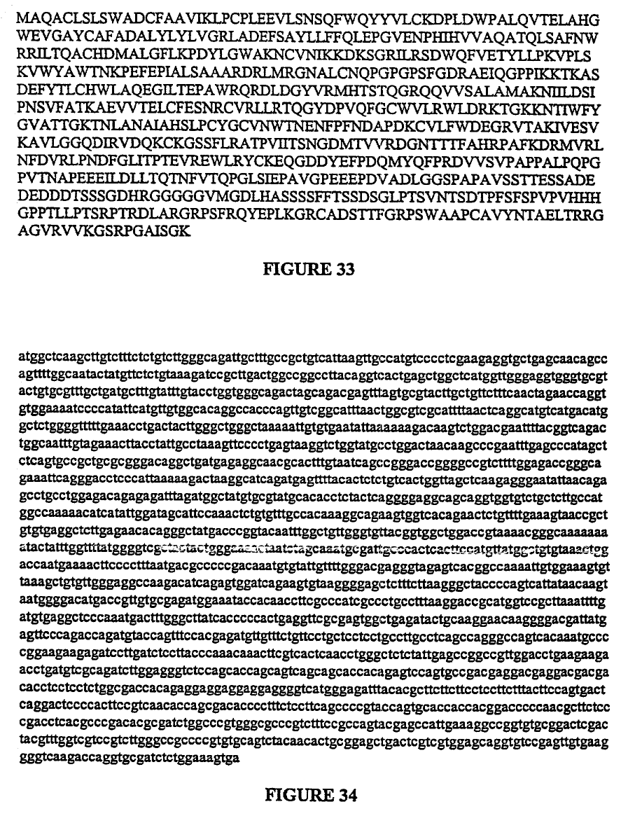

FIG. 33 depicts the nucleotide sequence (SEQ ID NO:39) of non-structural protein 1 isolated from chipmunk parvovirus.

FIG. 34 depicts the amino acid sequence (SEQ ID NO:40) of non-structural protein 1 isolated from chipmunk parvovirus.

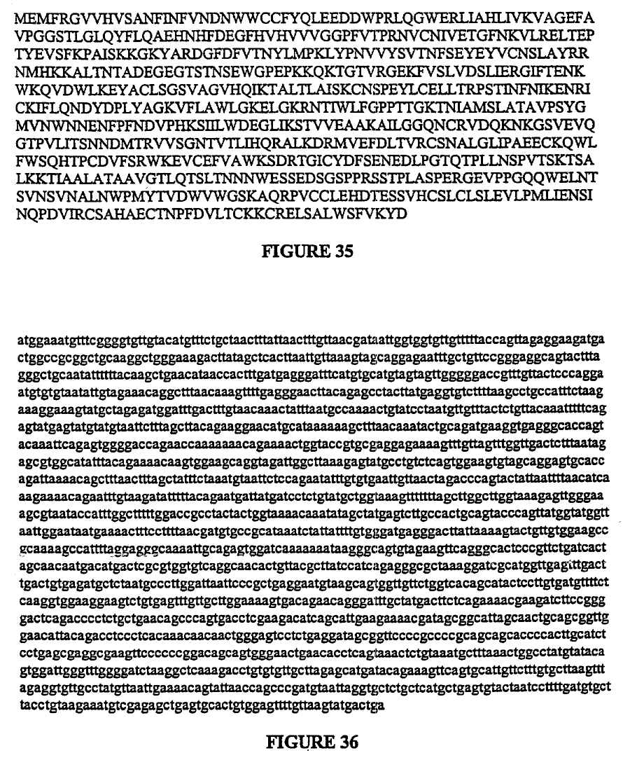

FIG. 35 depicts the nucleotide sequence (SEQ ID NO:41) of non-structural protein isolated from the pig-tailed macaque parvovirus.

FIG. 36 depicts the amino acid sequence (SEQ ID NO:42) of non-structural protein isolated from the pig-tailed macaque parvovirus.

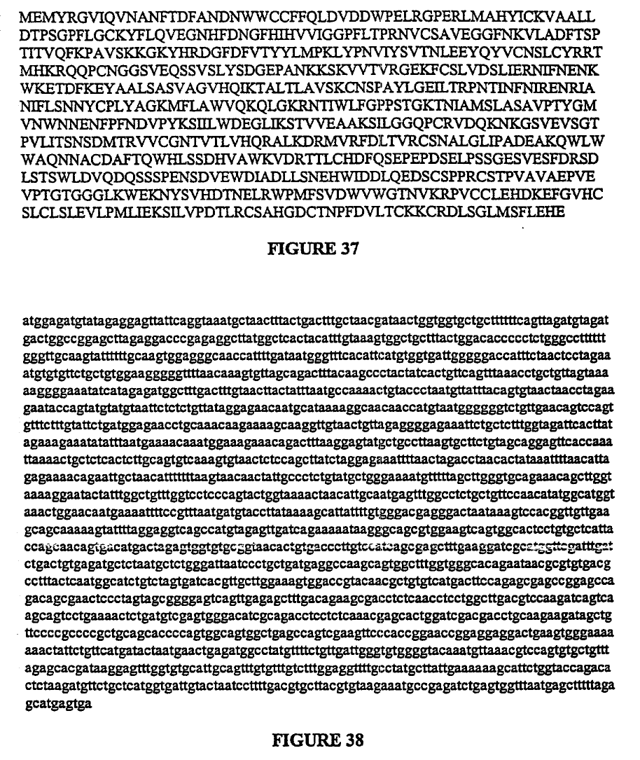

FIG. 37 depicts the nucleotide sequence (SEQ ID NO:43) of NS1 isolated from a simian parvovirus.

FIG. 38 depicts the amino acid sequence (SEQ ID NO:44) of NS1 protein isolated from a simian parvovirus.

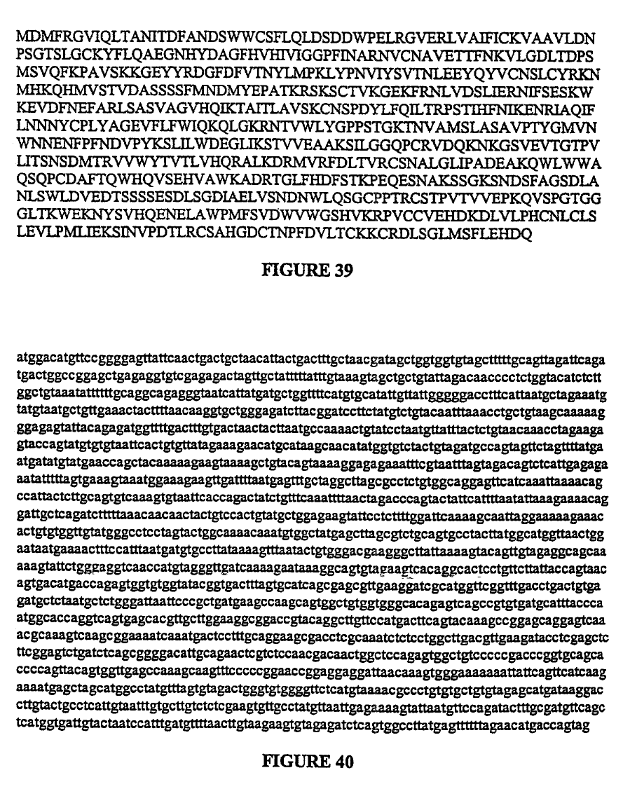

FIG. 39 depicts the nucleotide sequence (SEQ ID NO:45) of a NS protein isolated from the Rhesus macaque parvovirus.

FIG. 40 depicts the amino acid sequence (SEQ ID NO:46) of a NS protein isolated from the Rhesus macaque parvovirus.

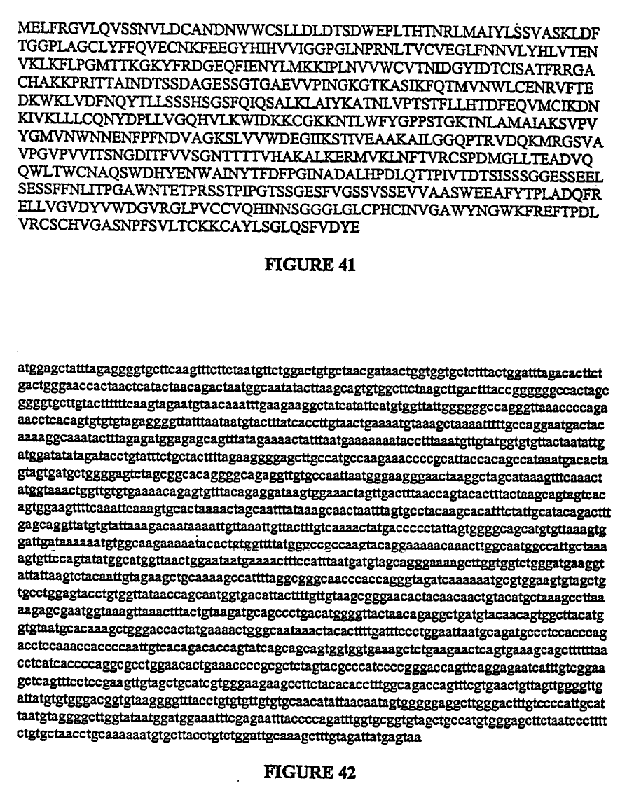

FIG. 41 depicts the nucleotide sequence (SEQ ID NO:47) of a non-structural protein isolated from the B19 virus.

FIG. 42 depicts the amino acid sequence (SEQ ID NO:48) of a non-structural protein isolated from the B19 virus.

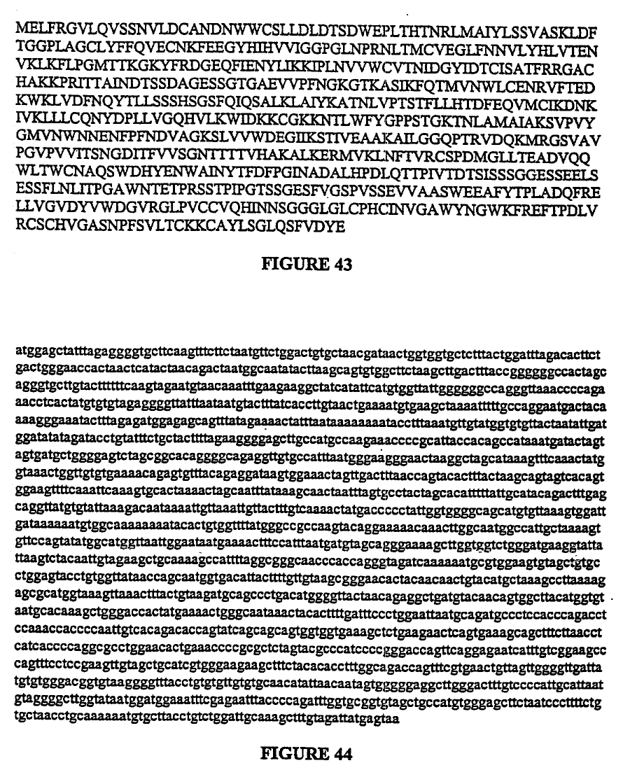

FIG. 43 depicts the nucleotide sequence (SEQ ID NO:49) of orf 1 isolated from the Erythrovirus B19.

FIG. 44 depicts the amino acid sequence (SEQ ID NO:50) of the product of orf 1 isolated from the Erythrovirus B19.

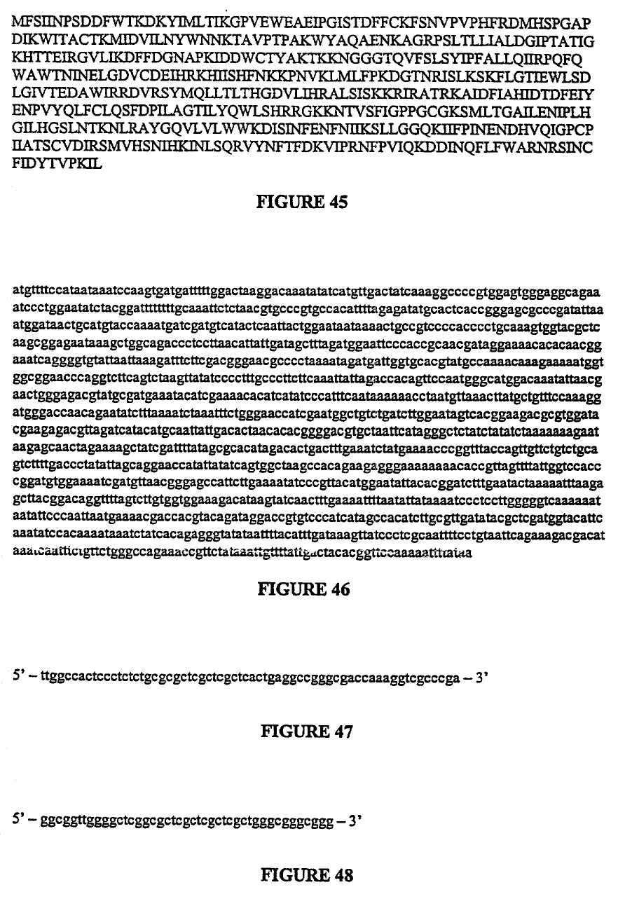

FIG. 45 depicts the nucleotide sequence (SEQ ID NO:51) of U94 isolated from the human herpesvirus 6B.

FIG. 46 depicts the amino acid sequence (SEQ ID NO:52) of U94 isolated from the human herpesvirus 6B.

FIG. 47 depicts an enzyme attachment site (SEQ ID NO:53) for a Rep protein.

FIG. 48 depicts the Rep 68 and Rep 78 enzyme attachment site (SEQ ID NO:54) found in chromosome 19.

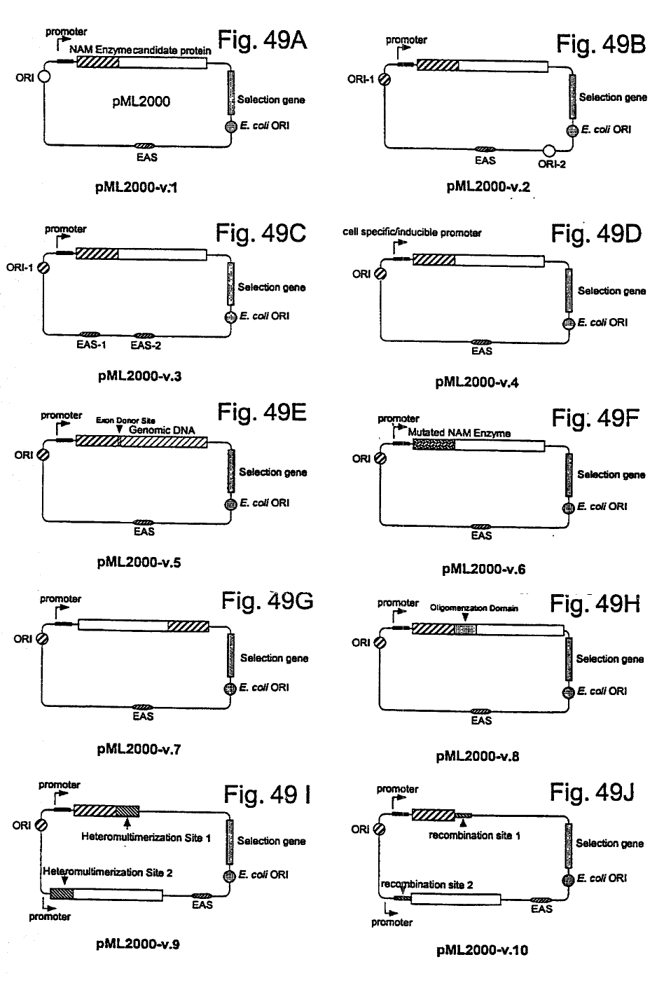

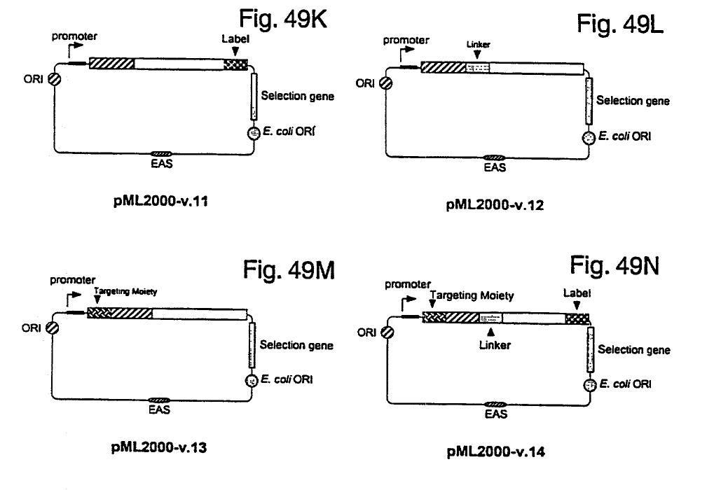

FIGS. 49A-49N depict preferred embodiments of the expression vectors of the invention.

### DETAILED DESCRIPTION OF THE INVENTION

Pharmaceutical compounds achieve their desired effects by interacting with target biomolecules such as proteins in a subject. These compounds often interact (i.e., cross react) with nontarget biomolecules also, causing unwanted side effects. This invention solves this problem by providing means for evaluating and predicting toxicity of a compound in a subject.

More particularly, this invention features a method of creating toxicity profiles of any given compound (or a combination of compounds) by using the compound(s) to screen nucleic acid expression libraries and identifying biomolecules (e.g., proteins) that this compound binds to. This in vitro information is then used to evaluate and predict the in vivo toxicity of the compound, based on the kinds of proteins the compound binds to and the tissue-specificity the binding has.

This toxicity profiling information can be used in many ways. For instance, it can be used to predict the toxicity (and efficacy) of a compound in different species, e.g., extrapolate the toxicity e ffects of a compound from one species to another. The information can also be used to predict toxicity of a compound in individuals of the same species that are under different physiological conditions (e.g., age, sex, and/or disease states), e.g., to identify individuals particularly susceptible to the toxicity. In a preferred embodiment, the new method is used to assess the safety of a candidate drug prior to a clinical trial. In a related embodiment, the new method is used to improve clinical trials by allowing the determination of toxic or unanticipated responses in humans early in a trial prior to overt tissue toxicity. In another embodiment, the new method helps evaluate and predict toxicity of chemicals in environmental or occupational settings such as in manufacturing and agriculture.

In yet another embodiment, the new method creates protein-interaction profiles for a panel of compounds. Such profiles are then correlated with the compounds' structures to create a SAR (structure-activity relationship) toxicity database and a predictive heuristic algorithm for toxicity. If these compounds share the same core structure, such a database will provide valuable information on how to minimize toxicity in rational drug design, e.g., how to improve a clinically efficacious but toxic drug. For these purposes, standard SAR techniques such as CoMFA (comparative molecular field analysis) and 3D QSAR (quantitative SAR) can be used. See, e.g., Garg et al., Crit Rev Toxicol 31:223-45 (2001); Lipnick, Environ Res 10:239-48 (1999); and Vedani et al., Prog Drug Res 55:105-35 (2000).

### I. EXEMPLARY COMPOUNDS

In this invention, toxicity profiles can be created for any synthetic or natural compound, including, without limitation, an organic or inorganic compound, a protein, a peptide, a nucleic acid, a metabolite, a drug derivative, or a chemical entity that is a hybrid form of the above. The compound can be a drug, a drug candidate, or an ingredient in human consumables (e.g., food, textile, cosmetics, flavors, fragrances, emulsifiers, surfactants, and detergents). The compound can also be any other substance that comes in contact with humans or other animals (e.g., pets and farm stock). They include, without limitation, pesticides, fertilizers, feed additives, antibiotics, herbicides, fungicides, polymer additives such as plasticizers, organic solvents, monomers, catalysts and crosslinkers, as well as environmental pollutants.

### II. EXPRESSION LIBRARIES

The nucleic acid expression libraries useful in this invention can be, without limitation, static cDNA libraries or conditional cDNA libraries. Static cDNA libraries are made from experimental animals on which toxicity tests are performed. Factors to consider when making such libraries include species, age and sex of the animals and the organ origin of the cDNA materials.

Conditional cDNA libraries can be made with mRNAs from tissues obtained upon, during, and/or after exposure of the animals or test cell lines to a biochemical perturbation, such as a chemical compound; or from tissues of animals under certain natural physiological conditions (e.g., pregnancy, viral infection, or diabetes) or under artificially created conditions that simulate the natural conditions. To make such libraries, it is important to consider the timing for harvesting the mRNAs. For instance, it may be desirable that the cellular hosts of the mRNAs are at a specific developmental or cell cycle stage, or at a certain time point after being treated with an agent. These conditions may provide insights into posttranslational states of the cells and/or expression of other accessory proteins required or useful for the desired purposes.

Regardless of the type of the expression library to be used, the method of this invention provides not only qualitative, but in many cases also quantitative, information on the binding between a test compound and its target proteins, because the abundance of a target protein in the expression library often correlates with the abundance of this protein in the tissue source of this library.

The libraries useful in this invention can also be made with genomic DNA. The libraries can be libraries made with naturally occurring nucleic acids or nonnaturally occurring nucleic acids (i.e., nucleic acids whose sequences do not exist in nature).

### III. TOXICOLOGY ANALYSES

The following describes exemplary embodiments in which toxicology analyses can be done in accordance with this invention.

The contact between a test compound and the expression library may be initiated prior to or after disruption of the membrane of the host cells (e.g., mammalian, insect or yeast cells). Complex formed between the test compound and the candidate proteins can then be isolated from the reaction mixture by using standard techniques. For instance, the test compound can be immobilized on a solid surface (e.g., a 96-well plate), and after the contacting step, any unbound proteins are washed away. Alternatively, the candidate proteins are bound to a solid surface, and after the contacting step, any unbound test compound are washed away. In yet another embodiment, either the test compound or the candidate proteins (or both) contain a moiety that can bind to a solid support (e.g., columns, microtiter plates, membranes, etc.). Such moiety include, without limitation, avidin, magnetic moieties, or moieties recognizable by a monoclonal antibody or having affinity for certain metals or ion exchange columns.

To detect the complexes formed between the test compound and candidate proteins, detectable moieties or moieties that can bind to a detectable label (e.g., peptide tags, fluorescent moieties, or radioactive moieties) can be attached or engineered into the test compound and/or the candidate protein.

To determine the binding affmity between the test compound and its bound protein ligand, binding assays well known in the art can be used. They include, without limitation, quantitative ELISA and competitive replacement assays (e.g., adding excess amounts of unlabeled test compound to a reaction mix containing complexes formed between radiolabeled test compound and candidate proteins). See also discussions below.

### A. Species-Specific Toxicology Tests

When the screening of this invention is done with expression libraries made from the same organ (e.g., liver, kidney, bone marrow, ovary, testes, or lymphoid tissues such as lymph nodes, thymus, or spleen) of different species, the screening results provide useful indices for the test compound in a given species, such as the presence or absence of a given interaction, the strength of the interaction, any developmental stage dependence of the interaction, or organ-specificity of the interaction.

Such information allows extrapolation of toxicity effects from one species to another based on the similarity of the toxicity profiles of the two species. Further, differences in the toxicity profiles between two species may help delineate why a given compound causes toxicity in one species but not another. These differences will be a suitable starting point for studying the toxicity mechanism.

One preferred organ for library construction is the liver, because the liver metabolizes most of the chemical compounds to which an organism is exposed and possess major detoxification pathways of an organism. Indeed, the levels of many liver cytochrome enzymes are used in ADME assays (see below) as toxicity indicators of a drug candidate.

The species-specific toxicity test can also help identify species-specific proteins that bind to a test compound. For instance, compound X is known to have serious toxicity in humans but not in mice. Screening of expression libraries made from the mouse and human livers may reveal novel proteins that interact with compound X and are present in the human but not mouse liver.

The species-specific test can also be used to compile protein-interaction profiles for species in which the toxicity of a given compound has been tested, and profiles for a species in which the toxicity of this compound has not been tested. The comparison of these two sets of profiles will help extrapolate toxicity effects from the tested species (e.g., murine) to an untested species (e.g., human).

### B. Differential Organ Interaction Tests

When the screening is done with expression libraries made from different tissues of the same species, the screening results provide useful indices concerning the organ-specific toxicity of the test compound. This organ-specific toxicity may be due to the ability of the compound to penetrate different organs (e.g., the ability to cross the blood brain barrier to reach the brain) and/or the existence of molecular targets in the susceptible organs. Such indices are useful in predicting the therapeutic or side effect (e.g., toxicity) of a drug. For instance, when a drug meant for treating a cardiovascular condition also binds strongly to kidney-specific proteins, one may predict that this drug can cause nephrotoxicity.

### C. Developmental Stage-Specific Toxicity Tests

When the screening is done with expression libraries made from the same tissue at different developmental stages, the screening results provide developmental stage-specific toxicity profiles. Such information is useful in predicting toxicity in subjects (e.g., humans) of different ages. See, e.g., Luster et al., Fundam Appl Toxicol 10:2-19 (1988).

### D. Individualized toxicity tests

The screening method of this invention can also be performed using expression libraries prepared from one organ (e.g., the liver) from a large number of individuals with different genetic background. The resultant information may be used to create a database for predicting how individuals with a particular genetic background may react to a compound, such as a drug or an environmental pollutant.

The individualized toxicity profile may be used in conjunction with the pharmacogenetic SNP (single nucleotide polymorphism) profile of an individual to better assess his risk of suffering toxicity from a particular compound. SNPs in a population confer survival advantages evolutionarily. But when combined with other polymorphisms or environmental factors, an SNP may become a low-level disease risk factor or disease modifier, and SNPs in genes encoding drug targets or drug metabolism pathways can determine the therapeutic utility and toxicity of drugs. Pharmacogenetic information based on SNPs can thus provide complementary information to the toxicity profile of a drug (or any other compound of interest) obtained in this invention. See, e.g., Haberkorn et al., European Journal of Nuclear Medicine 29:115-132 (2002); Diasio et al., Pharmacology 61:199-203 (2000); and Michelson et al., Curr Opin Mol Ther 2:651-654 (2000).

### E. Identification of Toxicity Pathways

The toxicity profiling information obtained in accordance with this invention can help identify the species-, tissue-, developmental stage-specific signaling pathway through which the test compound effects its intended function or unintended side effects.

For example, association of a given toxic endpoint (e.g., carcinogenicity, genotoxicity, and hepatoxicity) with a particular pattern of toxicant/protein binding observed in the library screening may provide a “fingerprint” that is characteristic of a specific mechanism of induction of that toxicity. Once a series of fingerprints is defined for different mechanisms, the protein-binding pattern of a compound of unknown toxicity can then be compared to the established patterns of defined mechanisms, yielding predictive information on the toxicity level and toxicity (as well as efficacy) mechanism of the new compound. See also Afshari, supra, Aardema et al., Mutational Research 499:13-25 (2002) and Olden et al., American Journal ofPublic Health 91:1964-1967 (2001), which discuss the parallel toxicogenomic approach linking toxicity with gene expression patterns.

By way of example, when a compound's toxicity profiles indicate that it not only binds to a target receptor in intended tissue A, but also binds to proteins X, Y, and Z in tissue B, the knowledge on the biological functions of proteins X, Y and Z may shed light on the mechanism by which the compound effects its toxicity in tissue B. In addition, a different compound showing a similar protein-binding pattern may be predicted as posing toxicity risk similar to that of the first compound.

The method of this invention can also be used to elucidate the defense mechanisms of an organism against a toxicant. For instance, a library screening of a toxicant is done with expression libraries prepared from tissue sources before and after exposure to the toxicant. The differences in the protein-binding profiles obtained with the two libraries may reveal the up regulation of enzymes involved in metabolizing the toxicant.

### F. Combination with other indexing tools

Other indexing tools or databases can also be used to enhance the utility and accuracy of the toxicity profiles obtained in the above described methods. These other indexing tools include the ADME assays required for FDA drug approval, and any other traditional toxicological, biochemical, immunohistochemical and animal assays. These tools also include expression profiling using DNA or RNA microarrays (gene chips), proteomic database of proteins or protein fragments, and yeast gene knock out database. See, e.g., Aardema et al. and Olden et al., supra; Bartosiewicz et al., Archives of Biochemistry and Biophysics 376:66-73 (2000), which describes the use of toxicological gene arrays containing DNA sequences for xenobiotic metabolizing enzymes, DNA repair enzymes, heat shock proteins, etc. to quantitatively assess mouse gene expression in response to β-naphthoflavone; Steiner et al., Annals of the New York Academy of Sciences 919:48-51 (2000), which describes using proteomics to elucidate drug toxicity pathways (cyclosporine A's nephrotoxicity) and to select pharmaceutical lead compounds; and Winzeler et al., Science 285:901-906 (1999), which describes gene deletional analysis of the S. cerevisiae genome.

### IV. PREFERRED EXPRESSION LIBRARIES

One problem facing high throughput library screening technologies today is the difficulty of elucidating the identification of the “hit,” i.e., a molecule causing the desired effect, against a background of many candidates that do not cause the desired effect. The PROCODE™ expression libraries described in WO 01/14539 (the disclosure of which is incorporated herein by reference in its entirety) circumvent this problem, and are therefore preferred expression libraries for the methods of this invention.

The PROCODE™ expression libraries rely on the use of nucleic acid modification enzymes that covalently and specifically bind to the nucleic acid molecules comprising the sequence that encodes them. Proteins of interest (for example, candidates to be screened for binding to a toxicant) are fused (either directly or indirectly, as outlined below) to a nucleic acid modification (NAM) enzyme. The NAM enzyme will covalently attach itself to a corresponding NAM attachment sequence (termed an enzyme attachment sequence (EAS)). Thus, by using vectors that comprise coding regions for the NAM enzyme and candidate proteins and the NAM enzyme attachment sequence, the candidate protein is covalently linked to the nucleic acid that encodes it upon translation in the host cell. Thus, after screening, candidates that bind to a test compound can be quickly isolated and identified using a variety of methods such as PCR amplification. This facilitates the quick identification of useful candidate proteins, and allows rapid screening and validation to occur.

Thus, each PROCODE™ expression vector comprises a “fusion nucleic acid” that encodes the NAM-candidate fusion protein. By “fusion protein” or grammatical equivalents herein is meant a protein composed of a plurality of protein components that while typically unjoined in their native state, are joined by their respective amino and/or carboxyl termini through a peptide linkage to form a single continuous polypeptide. The protein components can be joined directly (i.e., head to tail) or joined through a peptide linker/spacer as outlined below. Additional fusion partners, such as moieties that facility purification can also be included in the fusion protein (e.g., peptide tags such as histine tag (for binding to nickel); FLAG and myc tag (for binding to monoclonal antibodies); or Ig constant regions (for binding to protein A or Fc receptors).

### A. Nucleic Acid Modification Enzymes

The NAM enzyme useful in this invention is an enzyme that utilizes nucleic acids, particularly DNA, as a substrate and covalently attaches itself to a nucleic acid enzyme attachment sequence (EAS). The covalent attachment can be to the base, to the ribose moiety, or to the phosphate moieties. Many DNA binding proteins are known, such as those involved in nucleic acid compaction, transcription regulation and the like. But enzymes that covalently attach to nucleic acids, in particular enzymes or other proteins involved in nucleic acid replication, are generally preferred. Some NAM enzymes can form covalent linkages with DNA without nicking the DNA. For example, it is believed that enzymes involved in DNA repair recognize and covalently attach to nucleic acid regions that can be either double-stranded or single-stranded. Such NAM enzymes are suitable for use in the fusion library of this invention. However, NAM enzymes that nick DNA to form a covalent linkage (e.g., viral replication proteins) are also suitable.

Some DNA-binding enzymes form induced covalent linkage with nucleic acids upon physical or chemical stimulation, such as UV-induced crosslinking between DNA and a bound protein, or camptothecin (CPT)—related chemically induced trapping of the DNA-topoisomerase I covalent complex (e.g., Hertzberg et al., J. Biol. Chem. 265:19287-19295 (1990)). Such NAM enzymes are also suitable for use in some embodiments of the present invention.

In one embodiment, the NAM enzyme is a protein that recognizes specific nucleic acid sequences and/or conformations and performs its enzymatic activity such that a covalent complex is formed with the nucleic acid substrate. Preferably, the enzyme acts upon nucleic acids, particularly DNA, in various configurations including, but not limited to, single-stranded DNA, double-stranded DNA, Z-form DNA and the like.

Useful NAM enzymes include, but are not limited to, helicases, topoisomerases, polymerases, gyrases, recombinases, transposases, restriction enzymes and nucleases. NAM enzymes include natural and non-natural variants. Exemplary NAM enzymes are Rep proteins, NS 1 and H-1 of parvoviruses, bacteriophage phi-29 terminal proteins, the 55 Kd adenovirus proteins, and derivatives thereof.

1. REP proteins

In a preferred embodiment, the NAM enzyme is a Rep protein, such as Rep78 and Rep68 of of adeno-associated viruses (AAV), or a functional homolog found in related viruses. Rep proteins and their homologs may be found in a variety of sources including parvoviruses, erythroviruses, herpesviruses, and other related viruses. A naturally occurring Rep protein can be mutated or otherwise engineered to improve its activity and/or reduce its potential toxicity. Such improvements may be done in conjunction with their corresponding EAS.

One preferred Rep protein is an AAV Rep protein, which is encoded by the left open reading frame of the viral genome. AAV Rep proteins (e.g., Rep 78 and Rep68) regulate AAV transcription, activate AAV replication, and have been shown to inhibit transcription of heterologous promoters (Chiorini et al., J. ViroL. 68(2):797-804 (1994)). Rep68 and Rep78 act in part by covalently attaching to the AAV inverted terminal repeat (Prasad et al., Virology 229:183-192 (1997); Prasad et al., Virology 214:360 (1995)). They first generate a site-specific and strand-specific endonuclease nick at the AAV origin at the terminal resolution site and then covalently attach themselves to the 5′ terminus of the nicked site via a putative tyrosine linkage. Rep68 and Rep78 are produced by alternate splicing of the same transcript. The nucleic acid and protein sequences of Rep68 are shown in FIGS. 15 and 16, respectively. The nucleic acid and protein sequences of Rep78 proteins isolated from various sources are shown in FIGS. 1, 2, 7, 8, 13, and 14. The EAS' for Rep68 and Rep78 are shown in FIGS. 47 and 48, respectively, and discussed in Example 1.

The nucleic acid and amino acid sequences of other Rep homologs that are suitable for use as NAM enzymes are set forth in FIGS. 3-6, 17-28, 35, 36, and 39-46.

Functional fragments, variants, and homologs of naturally occurring Rep proteins are also included within the definition of Rep proteins. The variants/homologs possess nucleic acid binding activity and endonuclease activity.

2. NS1

In another preferred embodiment, the NAM enzyme is NS 1. NS 1 is a non-structural protein in parvovirus and a functional homolog of Rep78. It can also covalently attach to DNA (Cotmore et al., J. Virol. 62(3), 851-860 (1998)). The nucleotide and amino acid sequences of NS 1 proteins isolated from various sources are shown in FIGS. 9-12, 29-34, 37, and 38. Fragments and variants of NS1 proteins are also included within the definition of NS 1 proteins.

3. H-1 Protein

In a preferred embodiment, the NAM enzyme is the parvoviral H-1 protein, which is also known to form a covalent linkage with DNA. See, e.g., Tseng et al., Proc. Natl. Acad. Sci. USA 76(11):5539-5543 (1979). Fragments and variants of H-1 proteins are also included within the definition of H-1 proteins.

4. Bacteriophage phi-29 Terminal Protein

In a preferred embodiment, the NAM enzyme is the bacteriophage phi-29 terminal protein, which is also known to form a covalent linkage with DNA. See, e.g., Germendia et al., Nucleic Acid Research 16(3):5727-5740 (1988). Fragments and variants of phi-29 proteins are also included within the definition of phi-29 proteins.

5. a55 Protein

The NAM enzyme can also be the adenoviral 55 Kd (a55) protein, again known to form covalent linkage with DNA. See, e.g., Desiderio et al., J. Mol. Biol. 98:319-337 (1981). Fragments and variants of a55 proteins are also included within the definition of a55 proteins.

6. NAM Variants

Also included with the definition of NAM enzymes of the PROCODE™ libraries are amino acid sequence variants retaining biological activity (e.g., the ability to covalently attach to nucleic acid molecules) of naturally occurring NAMs or those NAMs that are specifically described herein. These variants fall into one or more of the following classes: substitutional, insertional or deletional variants. These variants can be prepared by site specific mutagenesis in the NAM encoding sequence, using cassette or PCR mutagenesis or other techniques well known in the art, to produce DNA encoding the variant, and thereafter expressing the recombinant DNA in host cells. Variant NAMs having up to about 100-150 residues may also be prepared by in vitro synthesis or peptide ligation using established techniques. Amino acid sequence variants are characterized by the predetermined nature of the variation, a feature that sets them apart from naturally occurring allelic or interspecies variation of the NAM protein amino acid sequence. The variants typically exhibit the same qualitative biological activity as the naturally occurring NAM enzyme, although variants can also be selected for having modified characteristics as will be more fully outlined below.

While the site or region for introducing an amino acid sequence variation is predetermined, the mutation per se need not be predetermined. For example, in order to optimize the performance, of a mutation at a given site, random mutagenesis may be conducted at the target codon or region and the expressed NAM variants screened for the optimal combination of desired activity. Techniques for making substitution mutations at predetermined sites in DNA having a known sequence are well known, for example, M13 primer mutagenesis and PCR mutagenesis. Screening of the mutants, variants, homologs, etc., is accomplished using assays of NAM enzymatic activities employing routine methods such as binding assays, affinity assays, peptide conformation mapping, and the like.

Amino acid substitutions are typically of single residues; insertions usually will be in the order of from 1 to about 20 amino acids, although considerably larger insertions may be tolerated. Deletions range from 1 to about 20 residues, although in some cases deletions may be much larger, for example when unnecessary domains are removed.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the NAM protein are desired, substitutions are generally made in accordance with the following chart:

Chart I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| PheSer | Met, Leu, Tyr |
| Thr | Thr |
| Trp | Ser |
| Tyr | Tyr |
| Val | Trp, Phe |
| | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the α-helical or β-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The variants typically exhibit the same qualitative biological activity as the naturally-occurring NAM enzyme, although variants also are selected to modify the characteristics of the NAM proteins as needed. Alternatively, the variant may be designed such that the biological activity of the NAM protein is altered. For example, glycosylation sites may be altered or removed. Similarly, functional mutations within the endonuclease domain or nucleic acid recognition site may be made. Furthermore, unnecessary domains may be deleted, to form fragments of NAM enzymes.

In addition, some embodiments utilize concatameric constructs to effect multivalency and increase binding kinetics or efficiency. For example, constructs containing a plurality of NAM coding regions or a plurality of EASs may be made.

Also included with the definition of NAM protein are other NAM homologs, and NAM proteins from other organisms including viruses, which are cloned and expressed as known in the art. Thus, probe or degenerate polymerase chain reaction (PCR) primer sequences may be used to find other related NAM proteins. As will be appreciated by those in the art, particularly useful probe and/or PCR primer sequences include the unique areas of the NAM nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

### B. Candidate Proteins

In addition to the NAM enzyme coding sequence, the fusion nucleic acid in the PROCODETM library also includes a coding sequence for a candidate protein whose binding to the compound of interest is to be tested.

Besides its potential to bind a test compound, the candidate protein may be engineered to possess an additional feature so as to allow ready retrieval of the desired NAP conjugates from the fusion protein library. For example, this feature is the ability of the candidate protein to mediate binding to a partner, the ability to alter cell phenotype (e.g., enzymatic activity such as β-galactosidase, luciferase, green fluorescent protein, proteinase, β-lactamase; or proteins that cause the cells to grow or to quench fluorescence) and structural or other physical properties including, but not limited to, electromagnetic behavior or spectroscopic behavior of the fusion proteins.

In a preferred embodiment, the candidate proteins are derived from cDNA libraries. The cDNA libraries may be complete libraries or partial libraries. Furthermore, the library of candidate proteins can be derived from a single cDNA source or multiple sources; that is, cDNA from multiple cell types or multiple individuals or multiple pathogens can be combined in a screen. The cDNA library may utilize entire cDNA constructs or fractionated constructs, including random or targeted fractionation. Suitable fractionation techniques include enzymatic, chemical or mechanical fractionation.

In another embodiment, the candidate proteins are derived from genomic libraries. The genomic libraries may be complete libraries or partial libraries. Furthermore, the library of candidate proteins can be derived from a single genomic source or multiple sources; that is, genomic DNA from multiple cell types or multiple individuals or multiple pathogens can be combined in a screen. The genomic library may utilize entire genomic constructs or fractionated constructs, including random or targeted fractionation. Suitable fractionation techniques include enzymatic, chemical or mechanical fractionation. In this embodiment, the fusion nucleic acid may comprise a splice donor sequence or splice acceptor sequence located between the NAM enzyme coding sequence and the genomic DNA. The incorporation of splice donor and/or splice acceptor sequences into the fusion nucleic acid sequence allows formation of a transcript encoding the NAM enzyme and exons of the genomic DNA fragment. Appropriate regulatory sequences can also be incorporated into the fusion nucleic acid molecule.

The candidate proteins may vary in size. In the case of cDNA or genomic libraries, the proteins may range from 20 or 30 amino acids to thousands, with from about 50 to 1000 (e.g., 75, 150, 350, 750 or more) being preferred and from 100 to 500 (e.g., 200, 300, or 400) being especially preferred. When the candidate proteins are peptides, the peptides are from about 3 to about 50 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In a preferred embodiment, libraries of candidate proteins are fused to the NAM enzymes, with each member of the library comprising a different candidate protein. However, as will be appreciated by those in the art, different members of the library may be reproduced or duplicated, resulting in some libraries members being identical. The library should provide a sufficiently structurally diverse population of expression products to effect a probabilistically sufficient range of cellular responses to provide one or more cells exhibiting a desired response. Accordingly, an interaction library must be large enough so that at least one of its members will have a structure that gives it affinity for some molecule, including both protein and non-protein targets, or other factors whose activity is necessary or effective within the assay of interest. Although it can be difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length has the potential to code for 20⁷ (10⁹) to 20²⁰. Thus, with libraries of 10⁷ to 10⁸ the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different expression products are simultaneously analyzed in the subject methods, although libraries of less complexity (e.g., 10², 10³, 10⁴ or 10⁵ different expression products) or greater complexity (e.g., 10¹⁰, 10¹¹, or 10¹² different expression products) are appropriate for use in the present invention. Preferred methods maximize library size and diversity.

In any library system encoded by oligonucleotide synthesis, complete control over the codons that will eventually be incorporated into the peptide structure is difficult. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is a high likelihood that 46.7% of the peptides will prematurely terminate. One way to alleviate this is to have random residues encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 15.6% chance to terminate prematurely. Alternatively, fusing the candidate proteins to the C-terminus of the NAM enzyme also may be done, although in some instances, fusing to the N-terminus means that prematurely terminating proteins result in a lack of NAM enzyme which eliminates these samples from the assay.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, PDZ domains, serines, threonines, tyrosines or histidines for phosphorylation sites, etc., or to purines, etc.

In a preferred embodiment, the bias is towards peptides or nucleic acids that interact with known classes of molecules. For example, when the candidate protein is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as pseudo substrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate proteins as well.

Thus, a number of molecules or protein domains are suitable as starting points for the generation of biased randomized candidate proteins. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

In a preferred embodiment, biased SH-3 domain-binding oligonucleotides/peptides are made. SH-3 domains have been shown to recognize short target motifs (SH-3 domain-binding peptides), about ten to twelve residues in a linear sequence, that can be encoded as short peptides with high affinity for the target SH-3 domain. Consensus sequences for SH-3 domain binding proteins have been proposed. Thus, in a preferred embodiment, oligos/peptides are made with the following biases:

```
                            1. XXXPPXPXX (SEQ ID NO: 1), wherein X is a randomized residue.
                            2. (within the positions of residue positions 11 to −2):
                                              11 10 9 8 7 6 5 4 3 2 1
                            Met Gly aa11 aa10 aa9 aa8 aa7 Arg Pro Leu Pro Pro hyd
                            0 −1 −2
                            Pro hyd hyd Gly Gly Pro Pro STOP (SEQ ID NO:2)
                            atg ggc nnk nnk nnk nnk nnk aga cct ctg cct cca sbk ggg sbk sbk gga
ggc cca cct TAA1 (SEQ ID NO:3).
```

In this embodiment, the N-terminus flanking region is suggested to have the greatest effects on binding affinity and is therefore entirely randomized. “Hyd” indicates a bias toward a hydrophobic residue, i.e.- Val, Ala, Gly, Leu, Pro, Arg. To encode a hydrophobically biased residue, “sbk” codon biased structure is used. Examination of the codons within the genetic code will ensure this encodes generally hydrophobic residues. s=g,c; b=t, g, c; v=a, g, c; m=a, c; k=t, g; n=a, t, g, c.

Thus, in a preferred embodiment, the candidate protein is a structural tag that will allow the isolation of target proteins with that structure. That is, in the case of leucine zippers, the fusion of the NAM enzyme to a leucine zipper sequence will allow the fusions to “zip up” with other leucine zippers, allow the quick isolation of a plurality of leucine zipper proteins. In addition, structural tags (which may only be the proteins themselves) can allow heteromultimeric protein complexes to form, that then are assayed for activity as complexes. That is, many proteins, such as many eucaryotic transcription factors, function as heteromultimeric complexes which can be assayed using the present invention.

In addition, rather than a cDNA, genomic, or random library, the candidate protein library may be a constructed library; that is, it may be built to contain only members of a defmed class, or combinations of classes. For example, libraries of immunoglobulins may be built, or libraries of G-protein coupled receptors, tumor suppressor genes, proteases, transcription factors, phosphatases, kinases, etc.

The fusion nucleic acid can comprise the NAM enzyme and candidate protein in a variety of configurations, including both direct and indirect fusions, and include N- and C-terminal fusions and internal fusions.

In a preferred embodiment, the NAM enzyme and the candidate protein are directly fused. In this embodiment, a direct, in-frame fusion of the nucleic acid encoding the NAM enzyme and the candidate protein is engineered. The library of fusion peptides can be constructed as N- and/or C-terminal fusions and internal fusions. Thus, the NAM enzyme coding region may be 3′ or 5′ to the candidate protein coding region, or the candidate protein coding region may be inserted into a suitable position within the coding region of the NAM enzyme. In this embodiment, it may be desirable to insert the candidate protein into an external loop of the NAM enzyme, either as a direct insertion or with the replacement of several of the NAM enzyme residues. This may be particularly desirable in the case of random candidate proteins, as they frequently require some sort of scaffold or presentation structure to confer a conformationally restricted structure. For an example of this general idea using green fluorescent protein (GFP) as a scaffold for the expression of random peptide libraries, see for example WO 99/20574.

### C. Linkers Between NAM And Candidate Proteins

The NAM enzyme and the candidate protein may be indirectly fused. This may be accomplished such that the components of the fusion remain attached, such as through the use of linkers, or in ways that result in the components of the fusion becoming separated. As will be appreciated by those in the art, there are a wide variety of different types of linkers that may be used, including cleavable and non-cleavable linkers; this cleavage may also occur at the level of the nucleic acid, or at the protein level.

In a preferred embodiment, linkers may be used to functionally isolate the NAM enzyme and the candidate protein. That is, a direct fusion system may sterically or functionally hinder the interaction of the candidate protein with its intended binding partner, and thus fusion configurations that allow greater degrees of freedom are useful. An analogy is seen in the single chain antibody area, where the incorporation of a linker allows functionality.

In a preferred embodiment, linkers known to confer flexibility are used. For example, useful linkers include glycine-serine polymers (including, for example, (GS)ₙ, and (GGGS)ₙ (SEQ ID NO:4) where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

The linker used to construct indirect fusion enzymes can be a cleavable linker. Cleavable linkers can function at the level of the nucleic acid or the protein. That is, cleavage (which in this sense means that the NAM enzyme and the candidate protein are separated) can occur during transcription, or before or after translation.

With respect to cleavable linkers, the cleavage can occur as a result of a cleavage functionality built into the nucleic acid. In this embodiment, for example, cleavable nucleic acid sequences, or sequences that will disrupt the nucleic acid, can be used. For example, intron sequences that the cell will remove can be placed between the coding region of the NAM enzyme and the candidate protein. In a preferred embodiment, the linkers are heterodimerization domains. In this embodiment, both the NAM enzyme and the candidate protein are fused to heterodimerization domains (or multimeric domains, if multivalency is desired), to allow association of these two proteins after translation.

In a preferred embodiment, cleavable protein linkers are used. In this embodiment, the fusion nucleic acids include coding sequences for a protein sequence that may be subsequently cleaved, generally by a protease. As will be appreciated by those in the art, cleavage sites directed to ubiquitous proteases, e.g. those that are constitutively present in most or all of the host cells of the system, can be used. Alternatively, cleavage sites that correspond to cell-specific proteases may be used. Similarly, cleavage sites for proteases that are induced only during certain cell cycles or phases or are signal specific events may be used as well.

There are a wide variety of possible proteinaceous cleavage sites known. For example, sequences that are recognized and cleaved by a protease or cleaved after exposure to certain chemicals are considered cleavable linkers. This may find particular use in in vitro systems, outlined below, as exogeneous enzymes can be added to the milieu or the NAP conjugates may be purified and the cleavage agents added. For example, cleavable linkers include, but are not limited to, the prosequence of bovine chymosin, the prosequence of subtilisin, the 2a site (Ryan et al., J. Gen. Virol. 72:2727 (1991); Ryan et al., EMBO J. 13:928 (1994); Donnelly et al., J. Gen. Virol. 78:13 (1997); Hellen et al., Biochem, 28(26):9881 (1989); and Mattion et al., J. Virol. 70:8124 (1996)), prosequences of retroviral proteases including human immunodeficiency virus protease and sequences recognized and cleaved by trypsin (EP 578472, Takasuga et al., J. Biochem. 112(5)652 (1992)) factor Xa (Gardella et al., J. Biol. Chem. 265(26):15854 (1990), WO 9006370), collagenase (J03280893, Tajima et al., J. Ferment. Bioeng. 72(5):362 (1991), WO 9006370), clostripain (EP 578472), subtilisin (including mutant H64A subtilisin, Forsberg et al., J. Protein Chem. 10(5):517 (1991), chymosin, yeast KEX2 protease (Bourbonnais et al., J. Bio. Chem. 263(30):15342 (1988), thrombin (Forsberg et al., supra; Abath et al., BioTechniques 10(2):178 (1991)), Staphylococcus aureus V8 protease or similar endoproteinase-Glu-C to cleave after Glu residues (EP 578472, Ishizaki et al., Appl. Microbiol. Biotechnol. 36(4):483 (1992)), cleavage by NIa proteainase of tobacco etch virus (Parks et al., Anal. Biochem. 216(2):413 (1994)), endoproteinase-Lys-C (U.S. Pat. No. 4,414,332) and endoproteinase-Asp-N, Neisseria type 2 IgA protease (Pohlner et al., Bio/Technology 10(7):799-804 (1992)), soluble yeast endoproteinase yscF (EP 467839), chymotrypsin (Altman et al., Protein Eng. 4(5):593 (1991)), enteropeptidase (WO 9006370), lysostaphin, a polyglycine specific endoproteinase (EP 316748), and the like. See e.g. Marston, F.A.O. (1986) Biol. Chem. J. 240, 1-12. Particular amino acid sites that serve as chemical cleavage sites include, but are not limited to, methionine for cleavage by cyanogen bromide (Shen, PNAS USA 81:4627 (1984); Kempe et al., Gene 39:239 (1985); Kuliopulos et al., J. Am. Chem. Soc. 116:4599 (1994); Moks et al., Bio/Technology 5:379 (1987); Ray et al., Bio/Technology 11:64 (1993)), acid cleavage of an Asp-Pro bond (Wingender et al., J. Biol. Chem. 264(8):4367 (1989); Gram et al., Bio/Technology 12:1017 (1994)), and hydroxylamine cleavage at an Asn-Gly bond (Moks, supra).

### D. Other Fusion Partners

In addition to the NAM enzymes, candidate proteins, and linkers, the fusion nucleic acids can comprise additional coding sequences for other functionalities. As will be appreciated by those in the art, the discussion herein is directed to fusions of these other components to the fusion nucleic acids described herein; however, they can also be separate from the fusion protein and rather be a component of the expression vector comprising the fusion nucleic acid, as is generally outlined below.

Thus, in a preferred embodiment, the fusions are linked to a fusion partner. By “fusion partner” or “functional group” herein is meant a sequence that is associated with the candidate protein, that confers upon all members of the library in that class a common function or ability. Fusion partners can be heterologous (i.e. not native to the host cell), or synthetic (not native to any cell). Suitable fusion partners include, but are not limited to: a) presentation structures, as defined below, which provide the candidate proteins in a conformationally restricted or stable form, including hetero- or homodimerization or multimerization sequences; b) targeting sequences, defined below, which allow the localization of the candidate proteins into a subcellular or extracellular compartment or be incorporated into infected organisms, such as those infected by viruses or pathogens; c) rescue sequences as defined below, which allow the purification or isolation of the NAP conjugates; d) stability sequences, which confer stability or protection from degradation to the candidate protein or the nucleic acid encoding it, for example resistance to proteolytic degradation; e) linker sequences; or f) any combination of a), b), c), d), and e), as well as linker sequences as needed.

In a preferred embodiment, the fusion partner is a presentation structure. By “presentation structure” or grammatical equivalents herein is meant an amino acid sequence, which, when fused to candidate proteins, causes the candidate proteins to assume a conformationally restricted form. This is particularly useful when the candidate proteins are random, biased random or pseudorandom peptides. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems.

Thus, synthetic presentation structures, i.e. artificial polypeptides, are capable of presenting a randomized peptide as a conformationally-restricted domain. Generally such presentation structures comprise a first portion joined to the N-terminal end of the randomized peptide, and a second portion joined to the C-terminal end of the peptide; that is, the peptide is inserted into the presentation structure, although variations may be made, as outlined below. To increase the functional isolation of the randomized expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, dimerization sequences, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a preferred embodiment, the presentation structure is a coiled-coil structure, allowing the presentation of the randomized peptide on an exterior loop. See, for example, Myszka et al., Biochem. 33:2362-2373 (1994), hereby incorporated by reference, and FIG. 3). Using this system investigators have isolated peptides capable of high affinity interaction with the appropriate target. In general, coiled-coil structures allow for between 6 to 20 randomized positions. A preferred coiled-coil presentation structure is described in, for example, Martin et al., EMBO J. 13(22):5303-5309 (1994), incorporated by reference.

In a preferred embodiment, the presentation structure is a minibody structure. A “minibody” is essentially composed of a minimal antibody complementarity region. The minibody presentation structure generally provides two randomizing regions that in the folded protein are presented along a single face of the tertiary structure. See, for example, Bianchi et al., J. Mol. Biol. 236(2):649-59 (1994), and references cited therein, all of which are incorporated by reference. Investigators have shown this minimal domain is stable in solution and have used phage selection systems in combinatorial libraries to select minibodies with peptide regions exhibiting high affinity, Kd=10⁻⁷, for the pro-inflammatory cytokine IL-6.

A preferred minibody presentation structure is as follows: MGRNSQATSGFTFSHFYMEWVRGGEYIAASRHKHNKYTTEYSASVKGRYI VSRDTSQSILYLQKKKGPP (SEQ ID NO:5). The bold, underline regions are the regions which may be randomized. The italized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXI sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly crosslinked structured conformations, similar to a presentation structure. Similarly, the randomization regions may be controlled to contain a certain number of residues to confer β-sheet or α-helical structures.

In one embodiment, the presentation structure is a dimerization or multimerization sequence. A dimerization sequence allows the non-covalent association of one candidate protein to another candidate protein, including peptides, with sufficient affinity to remain associated under normal physiological conditions. This effectively allows small libraries of candidate protein (for example, 10⁴) to become large libraries if two proteins per cell are generated which then dimerize, to form an effective library of 10⁸ (10⁴=10⁴). It also allows the formation of longer proteins, if needed, or more structurally complex molecules. The dimers may be homo- or heterodimers.

Dimerization sequences may be a single sequence that self-aggregates, or two sequences. That is, nucleic acids encoding both a first candidate protein with dimerization sequence 1, and a second candidate protein with dimerization sequence 2, such that upon introduction into a cell and expression of the nucleic acid, dimerization sequence 1 associates with dimerization sequence 2 to form a new structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

In a preferred embodiment, the fusion partner is a targeting sequence. As will be appreciated by those in the art, the localization of proteins within a cell is a simple method for increasing effective concentration and determining function. For example, RAF1 when localized to the mitochondrial membrane can inhibit the antiapoptotic effect of BCL-2. Similarly, membrane bound Sos induces Ras mediated signaling in T-lymphocytes. These mechanisms are thought to rely on the principle of limiting the search space for ligands, that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller space thereby increasing concentration. Finally, the ligand or target may simply be localized to a specific compartment, and inhibitors must be localized appropriately.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane or within pathogens or viruses that have infected the cell; and b) extracellular locations via a secretory signal. Particularly preferred is localization to either subcellular locations or to the outside of the cell via secretion.

In a preferred embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509,; the human retinoic acid receptor-β nuclear localization signal; NFkB p50 (see, for example, Ghosh et al., Cell 62:1019 (1990)); NFKB p65 (see, for example, Nolan et al., Cell 64:961 (1991)); and others (see, for example, Boulikas, J. Cell. Biochem. 55(1):32-58 (1994), hereby incorporated by reference) and double basic NLS's exemplified by that of the Xenopus (African clawed toad) protein, nucleoplasmin (see, for example, Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

In a preferred embodiment, the targeting sequence is a membrane anchoring signal sequence. This is particularly useful since many parasites and pathogens bind to the membrane, in addition to the fact that many intracellular events originate at the plasma membrane. Thus, membrane-bound peptide libraries are useful for both the identification of important elements in these processes as well as for the discovery of effective inhibitors. In addition, many drugs interact with membrane associated proteins. The invention provides methods for presenting the candidate proteins extracellularly or in the cytoplasmic space. For extracellular presentation, a membrane anchoring region is provided at the carboxyl terminus of the candidate protein. The candidate protein region is expressed on the cell surface and presented to the extracellular space, such that it can bind to other surface molecules (affecting their function) or molecules present in the extracellular medium. The binding of such molecules could confer function on the cells expressing a peptide that binds the molecule. The cytoplasmic region could be neutral or could contain a domain that, when the extracellular candidate protein region is bound, confers a function on the cells (activation of a kinase, phosphatase, binding of other cellular components to effect function). Similarly, the candidate protein-containing region could be contained within a cytoplasmic region, and the transmembrane region and extracellular region remain constant or have a defined function.

In addition, it should be noted that in this embodiment, as well as others outlined herein, it is possible that the formation of the NAP conjugate happens after the screening; that is, having the fusion protein expressed on the extracellular surface means that it may not be available for binding to the nucleic acid. However, this may be done later, with lysis of the cell.

Membrane-anchoring sequences are well known in the art and are based on the genetic geometry of mammalian transmembrane molecules. Peptides are inserted into the membrane based on a signal sequence (designated herein as ssTM) and require a hydrophobic transmembrane domain (herein TM). The transmembrane proteins are inserted into the membrane such that the regions encoded 5′ of the transmembrane domain are extracellular and the sequences 3′ become intracellular. Of course, if these transmembrane domains are placed 5′ of the variable region, they will serve to anchor it as an intracellular domain, which may be desirable in some embodiments. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

Membrane-anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful membrane-anchoring sequences include, for example, sequences from: 1) class I integral membrane proteins such as IL-2 receptor betachain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the transmembrane domain and 960-1382 are the cytoplasmic domain; see Hatakeyama, supra, and Ebina et al., Cell 40:747 (1985)); 2) class II integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (see, for example, Nakauchi et al., PNAS USA 82:5126 (1985) and 1-21 in the case of ICAM-2 (see, for example, Staunton et al., Nature (London) 339:61 (1989)). These leader sequences deliver the construct to the membrane while the hydrophobic transmembrane domains, placed 3′ of the random candidate region, serve to anchor the construct in the membrane. These transmembrane domains are encompassed by amino acids 145-195 from CD8 (Nakauchi, supra) and 224-256 from ICAM-2 (Staunton, supra).

Alternatively, membrane anchoring sequences can include the GPI anchor, which results in a covalent bond between the molecule and the lipid bilayer via a glycosyl-phosphatidylinositol bond for example in DAF (see, for example, Homans et al., Nature 333(6170):269-72 (1988), and Moran et al., J. Biol. Chem. 266:1250 (1991)). In order to do this, the GPI sequence from Thy-1 can be inserted 3′ of the variable region in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function (see Cross et al., Mol. Cell. Biol. 4(9):1834 (1984); Spencer et al., Science 262:1019-1024 (1993), both of which are hereby incorporated by reference). This motif has already been shown to be effective in the localization of reporter genes and can be used to anchor the zeta chain of the TCR. This motif is placed 5′ of the variable region in order to localize the construct to the plasma membrane. Other modifications such as palmitoylation can be used to anchor constructs in the plasma membrane; for example, palmitoylation sequences from the G protein-coupled receptor kinase GRK6 sequence (see, for example, Stoffel et al., J. Biol. Chem 269:27791 (1994)); from rhodopsin (see, for example, Barnstable et al., J. Mol. Neurosci. 5(3):207 (1994)); and the p21 H-ras 1 protein (see, for example, Capon et al., Nature 302:33 (1983)).

In a preferred embodiment, the targeting sequence is a lysozomal targeting sequence, including, for example, a lysosomal degradation sequence such as Lamp-2 (KFERQ; Dice, Ann. N.Y. Acad. Sci. 674:58 (1992); or lysosomal membrane sequences from Lamp-1 (see, for example, Uthayakumar et al., Cell. Mol. Biol. Res. 41:405 (1995)) or Lamp-2 (see, for example, Konecki et la., Biochem. Biophys. Res. Comm. 205:1-5 (1994)).

Alternatively, the targeting sequence can comprise a mitrochondrial localization sequence, including mitochondrial matrix sequences (e.g. yeast alcohol dehydrogenase III; Schatz, Eur. J. Biochem. 165:1-6 (1987)); mitochondrial inner membrane sequences (yeast cytochrome c oxidase subunit IV; Schatz, supra); mitochondrial intermembrane space sequences (yeast cytochrome cl; Schatz, supra) or mitochondrial outer membrane sequences (yeast 70 kD outer membrane protein; Schatz, supra).

The target sequences also can comprise endoplasmic reticulum sequences, including the sequences from calreticulin (Pelham, Royal Society London Transactions B; 1-10 (1992)) or adenovirus E3/19K protein (see, for example, Jackson et al., EMBO J. 9:3153 (1990)).

Furthermore, targeting sequences also can include peroxisome sequences (for example, the peroxisome matrix sequence from Luciferase; Keller et al., PNAS USA 4:3264 (1987)); famesylation sequences (for example, P21 H-ras 1; Capon, supra); geranylgeranylation sequences (for example, protein rab-5A; Farnsworth, PNAS USA 91:11963 (1994)); or destruction sequences (cyclin B 1; Klotzbucher et al., EMBO J. 1:3053 (1996)).

In a preferred embodiment, the targeting sequence is a secretory signal sequence capable of effecting the secretion of the candidate protein. There are a large number of known secretory signal sequences which are placed 5′ to the variable peptide region, and are cleaved from the peptide region to effect secretion into the extracellular space. Secretory signal sequences and their transferability to unrelated proteins are well known, e.g., Silhavy, et al. (1985) Microbiol. Rev. 49, 398-418. This is particularly useful to generate a peptide capable of binding to the surface of, or affecting the physiology of, a target cell that is other than the host cell. In this manner, target cells grown in the vicinity of cells caused to express the library of peptides, are bathed in secreted peptide. Target cells exhibiting a physiological change in response to the presence of a peptide, e.g., by the peptide binding to a surface receptor or by being internalized and binding to intracellular targets, and the secreting cells are localized by any of a variety of selection schemes and the peptide causing the effect determined. Exemplary effects include variously that of a designer cytokine (i.e., a stem cell factor capable of causing hematopoietic stem cells to divide and maintain their totipotential), a factor causing cancer cells to undergo spontaneous apoptosis, a factor that binds to the cell surface of target cells and labels them specifically, etc.

Similar to the membrane-anchored embodiment, it is possible that the formation of the NAP conjugate happens after the screening; that is, having the fusion protein secreted means that it may not be available for binding to the nucleic acid. However, this may be done later, with lysis of the cell.

Suitable secretory sequences are known, including, for example, signals from IL-2 (see, for example, Villinger et al., J. Immunol. 155:3946 (1995)), growth hormone (see, for example, Roskam et al., Nucleic Acids Res. 7:30 (1979)); preproinsulin (see, for example, Bell et al., Nature 284:26 (1980)); and influenza HA protein (see, for example, Sekiwawa et al., PNAS 80:3563)). A particularly preferred secretory signal sequence is the signal leader sequence from the secreted cytokine IL-4.

In a preferred embodiment, the fusion partner is a rescue sequence (sometimes also referred to herein as “purification tags” or “retrieval properties”). A rescue sequence is a sequence which may be used to purify or isolate either the candidate protein or the NAP conjugate. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST. Rescue sequences can be utilized on the basis of a binding event, an enzymatic event, a physical property or a chemical property.

Alternatively, the rescue sequence can comprise a unique oligonucleotide sequence which serves as a probe target site to allow the quick and easy isolation of the construct, via PCR, related techniques, or hybridization.

In a preferred embodiment, the fusion partner is a stability sequence to confer stability to the candidate protein or the nucleic acid encoding it. Thus, for example, peptides can be stabilized by the incorporation of glycines after the initiation methionine, for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate protein structure. Thus, preferred stability sequences are as follows: MG(X)ₙ,GGPP, where X is any amino acid and n is an integer of at least four.

In addition, linker sequences, as defined above, may be used in any configuration as needed.

In addition, the fusion partners, including presentation structures, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

Combinations of fusion partners can be used if desired. Thus, for example, any number of combinations of presentation structures, targeting sequences, rescue sequences, and stability sequences may be used, with or without linker sequences. Similarly, as discussed herein, the fusion partners may be associated with any component of the expression vectors described herein: they may be directly fused with either the NAM enzyme, the candidate protein, or the EAS, described below, or be separate from these components and contained within the expression vector.

### E. EAS

In addition to sequences encoding NAM enzymes and candidate proteins, and the optional fusion partners, the nucleic acids of the PROCODE™ libraries preferably comprise an enzyme attachment sequence. By “enzyme attachment sequence” or “EAS” herein is meant selected nucleic acid sequences that mediate attachment with NAM enzymes. Such EAS nucleic acid sequences possess the specific sequence or specific chemical or structural configuration that allows for attachment of the NAM enzyme and the EAS. The EAS can comprise DNA or RNA sequences in their natural conformation, or hybrids. EASs also can comprise modified nucleic acid sequences or synthetic sequences inserted into the nucleic acid molecule of the present invention. EASs also can comprise non-natural bases or hybrid non-natural and natural (i.e., found in nature) bases.

As will be appreciated by those in the art, the choice of the EAS will depend on the NAM enzyme, as individual NAM enzymes recognize specific sequences and thus their use is paired. Thus, suitable NAM/EAS pairs are the sequences recognized by Rep proteins (sometimes referred to herein as “Rep EASs”) and the Rep proteins, the H-1 recognition sequence and H-1, etc. In addition, EASs can be utilized which mediate improved covalent binding with the NAM enzyme compared to the wild-type or naturally occurring EAS.

In a preferred embodiment, the EAS is double-stranded. By way of example, a suitable EAS is a double-stranded nucleic acid sequence containing specific features for interacting with corresponding NAM enzymes. For example, Rep68 and Rep78 recognize an EAS contained within an AAV ITR, the sequence of which is set forth in Example 1. In addition, these Rep proteins have been shown to recognize an ITR-like region in human chromosome 19 as well, the sequence of which is shown in FIG. 48.

An EAS also can comprise supercoiled DNA with which a topoisomerase interacts and forms covalent intermediate complexes. Alternatively, an EAS is a restriction enzyme site recognized by an altered restriction enzyme capable of forming covalent linkages. Finally, an EAS can comprise an RNA sequence and/or structure with which specific proteins interact and form stable complexes (see, for example, Romaniuk and Uhlenbeck, Biochemistry, 24, 4239-44 (1985)).

The present invention relies on the specific binding of the NAM enzyme to the EAS in order to mediate linkage of the fusion enzyme to the nucleic acid molecule. One of ordinary skill in the art will appreciate that use of an EAS consisting of a small nucleic acid sequence would result in non-specific binding of the NAM enzyme to expression vectors and the host cell genome depending on the frequency that the accessible EAS motif appears in the vector or host genome. Therefore, the EAS of the present invention is preferably comprised of a nucleic acid sequence of sufficient length such that specific fusion protein-coding nucleic acid molecule attachment results. For example, the EAS is preferably greater than five nucleotides in length. More preferably, the EAS is greater than 10 nucleotides in length, e.g., with EASs of at least 12, 15, 20, 25, 30, 35, 40, 45 or 50 nucleotides being preferred.

Moreover, preferably the EAS is present in the host cell genome in a very limited manner, such that at most, only one or two NAM enzymes can bind per genome, e.g. no more than once in a human cell genome. In situations wherein the EAS is present many times within a host cell, e.g., a human cell genome, the probability of fusion proteins encoded by the expression vector attaching to the host cell genome and not the expression vector increases and is therefore undesirable. For instance, the bacteriophage P2 A protein recognizes a relatively short DNA recognition sequence. As such, use of the P2 A protein in mammalian cells would result in protein binding throughout the host genome, and identification of the desired nucleic acid sequence would be difficult. Thus, preferred embodiments exclude the use of P2A as a NAM enzyme.

One of ordinary skill in the art will appreciate that the NAM enzyme used in the present invention or the corresponding EAS can be manipulated in order to increase the stability of the fusion protein-nucleic acid molecule complex. Such manipulations are contemplated herein, so long as the NAM enzyme forms a covalent bond with its corresponding EAS.

It can be advantageous to control attachment of the fusion enzyme to the EAS. For example, the EAS can be introduced into the nucleic acid molecule as two non-functional halves that are brought together following enzyme-mediated or non-enzyme-mediated homologous recombination, such as that mediated by cre-lox recombination, to form a functional EAS. Likewise, the referenced cre-lox consideration can also be used to control the formation of a functional fusion enzyme. The control of cre-lox recombination is preferably mediated by introducing the recombinase gene under the control of an inducible promoter into the expression system, whether on the same nucleic acid molecule or on another expression vector.

### F. Expression Vectors

Thus, in a preferred embodiment, the nucleic acids of the invention comprise (i) a fusion nucleic acid comprising sequences encoding a NAM enzyme and a candidate protein, and (ii) an EAS. These nucleic acids are preferably incorporated into an expression vector; thus providing libraries of expression vectors, sometimes referred to herein as “NAM enzyme expression vectors”.

The expression vectors may be either self-replicating extrachromosomal vectors, vectors which integrate into a host genome, or linear nucleic acids that may or may not self-replicate. Thus, specifically included within the definition of expression vectors are linear nucleic acid molecules. Expression vectors thus include plasmids, plasmid-liposome complexes, phage vectors, and viral vectors, e.g., adeno-associated virus (AAV)-based vectors, retroviral vectors, herpes simplex virus (HSV)-based vectors, and adenovirus-based vectors. The nucleic acid molecule and any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994) Generally, these expression vectors include transcriptional and translational regulatory nucleic acid sequences operably linked to the nucleic acid encoding the NAM protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the NAM protein, as will be appreciated by those iri the art; for example, transcriptional and translational regulatory nucleic acid sequences from Bacillus are preferably used to express the NAM protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer, silencer, or activator sequences. In a preferred embodiment, the regulatory sequences include a promoter and transcriptional start and stop sequences.

A “promoter” is a nucleic acid sequence that directs the binding of RNA polymerase and thereby promotes RNA synthesis. Promoter sequences include constitutive and inducible promoter sequences. Exemplary constitutive promoters include, but are not limited to, the CMV immediate-early promoter, the RSV long terminal repeat, mouse mammary tumor virus (MMTV) promoters, etc. Suitable inducible promoters include, but are not limited to, the IL-8 promoter, the metallothionine inducible promoter system, the bacterial 1acZYA expression system, the tetracycline expression system, and the T7 polymerases system. The promoters can be either naturally occurring promoters, hybrid promoters, or synthetic promoters. Hybrid promoters, which combine elements of more than one promoter, are also known in the art, and are useful in the present invention.

In addition, the expression vector may comprise additional elements. For example, the expression vector may have two replication systems (e.g., origins of replication), thus allowing it to be maintained in two organisms, for example in animal cells for expression and in a prokaryotic host for cloning and amplification. Furthermore, for integrating expression vectors, which are generally not preferred in most embodiments, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

It should be noted that the compositions and methods of the present invention allow for specific chromosomal isolation. For example, since human chromosome 19 contains a Rep-binding sequence (e.g., an EAS), a NAP conjugate will be formed with chromosome 19, when the NAM enzyme is Rep. Cell lysis followed by immunoprecipitation, either using antibodies to the Rep protein itself (e.g. no candidate protein is necessary) or to a fused candidate protein or purification tag, allows the purification of the chromosome. This is a significant advance over current chromosome purification techniques. Thus, by selectively or non-selectively integrating EAS sites into chromosomes, different chromosomes may be purified.

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers new phenotypes of the cells which contain the vector. These phenotypes include, for instance, enhanced or decreased cell growth. The phenotypes can also include resistance to a selection agent. Suitable selection agents include, but are not limited to, neomycin (or its analog G418), blasticidin S, histinidol D, bleomycin, puromycin, hygromycin B, and other drugs. The expression vector also can comprise a coding sequence for a marker protein, such as the green fluorescence protein, which enables, for example, rapid identification of successfully transduced cells.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

One expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al.,Proc. Natl. Acad. Sci. U.S.A., 93:5185-90; Choate et al., Human Gene Therapy, 7:2247 (1996); PCT/US97/01019 and PCT/US97/01048, and references cited therein.

The fusion proteins of the present invention can be produced by culturing a host cell transformed with nucleic acid, preferably an expression vector as outlined herein, under the appropriate conditions to induce or cause production of the fusion protein. The conditions appropriate for fusion protein production will vary with the choice of the expression vector and the host cell, and will be easily ascertained by one skilled in the art using routine methods. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cells are lytic viruses, and thus harvest time selection can be crucial for product yield.

Any host cell capable of withstanding introduction of exogenous DNA and subsequent protein production is suitable for the present invention. The choice of the host cell will depend, in part, on the assay to be run; e.g., in vitro systems may allow the use of any number of procaryotic or eucaryotic organisms, while ex vivo systems preferably utilize animal cells, particularly mammalian cells with a special emphasis on human cells. Thus, appropriate host cells include yeast, bacteria, archaebacteria, plant, and insect and animal cells, including mammalian cells and particularly human cells. The host cells may be native cells, primary cells, including those isolated from diseased tissues or organisms, cell lines (again those originating with diseased tissues), genetically altered cells, etc. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, etc. See the ATCC cell line catalog.

In a preferred embodiment, the fusion proteins are expressed in mammalian cells. Mammalian expression systems are also known in the art, and include, for example, retroviral and adenoviral systems. A mammalian promoter is any DNA sequence capable of binding mammalian RNA polymerase and initiating the downstream (3′) transcription of a coding sequence for a fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature MRNA is formed by site-specific post-translational cleavage and polyadenylation. Examples of transcription terminator and polyadenlytion signals include those derived from SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, NAM fusions are produced in bacterial systems. Bacterial expression systems are widely available and include, for example, plasmids.

A suitable bacterial promoter is any nucleic acid sequence capable of binding bacterial RNA polymerase and initiating the downstream (3′) transcription of the coding sequence of the fusion into MRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the fusion proteins in bacteria or other cells. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

Suitable bacterial vectors include, for example, vectors for Bacillus subtilis, E. coli, Streptococcus cremoris, and Streptococcus lividans, among others. The bacterial expression vectors can be transformed into bacterial host cells using techniques well known in the art, such as calcium chloride treatment, electroporation, and others. One benefit of using bacterial cells in the ability to propagate the cells comprising the expression vectors, thus generating clonal populations.

NAM fusion proteins also can be produced in insect cells such as Sf9 cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In addition, NAM fusion proteins can be produced in yeast cells. Yeast expression systems are well known in the art, and include, for example, expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphonate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions. One benefit of using yeast cells is the ability to propagate the cells comprising the vectors, thus generating clonal populations.

NAM fusion proteins can also be produced in mammalian cells. For instance, they can be produced in cells of mouse, rat, primate or human origin.

Some preferred expression vectors are shown in FIGS. 49A-49N.

In addition to the components outlined herein, including NAM enzyme-candidate protein fusions, EASs, linkers, fusion partners, etc., the expression vectors may comprise a number of additional components, including, selection genes as outlined herein (particularly including growth-promoting or growth-inhibiting functions), activatible elements, recombination signals (e.g. cre and lox sites) and labels.

### G. Detectable Labels

The NAP conjugates further comprise a labeling component. Again, as for the fusion partners of the invention, the label can be fused to one or more of the other components, for example to the NAM fusion protein, in the case where the NAM enzyme and the candidate protein remain attached, or to either component, in the case where scission occurs, or separately, under its own promoter. In addition, as is further described below, other components of the assay systems may be labeled.

Labels can be either direct or indirect detection labels, sometimes referred to herein as “primary” and “secondary” labels. By “detection label” or “detectable label” herein is meant a moiety that allows detection. This may be a primary label or a secondary label. Accordingly, detection labels may be primary labels (i.e. directly detectable) or secondary labels (indirectly detectable).

In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles. In a preferred embodiment, the detection label is a primary label. A primary label is one that can be directly detected, such as a fluorophore.

Preferred labels include, for example, chromophores or phosphors but are preferably fluorescent dyes or moieties. Fluorophores can be either “small molecule” fluors, or proteinaceous fluors. In a preferred embodiment, particularly for labeling of target molecules, as described below, suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland.

In a preferred embodiment, for example when the label is attached to the fusion polypeptide or is to be expressed as a component of the expression vector, proteinaceous fluores are used. Suitable autofluorescent proteins include, but are not limited to, the green fluorescent protein (GFP) from Aequorea and variants thereof (Cody et al., Biochemistry 32:1212-1218 (1993); and Inouye et al., FEBS Lett. 341:277-280 (1994)), including, but not limited to, GFP (Chalfie et al., Science 263(5148):802-805 (1994)), enhanced GFP (EGFP; Clontech-Genbank Accession Number U55762 )), blue fluorescent protein (BFP; Quantum Biotechnologies, Inc., 1801 de Maisonneuve Blvd. West, 8th Floor, Montreal (Quebec) Canada H3H 1J9; Stauber, Biotechniques 24(3):462-471 (1998); Heim et al., Curr. Biol. 6:178-182 (1996)), and enhanced yellow fluorescent protein (EYFP; Clontech Laboratories, Inc., 1020 East Meadow Circle, Palo Alto, Calif. 94303). In addition, autofluorescent proteins from Renilla species can also be used. See, e.g., WO 92/15673, WO 95/07463, WO 98/14605, WO 98/26277, WO 99/49019; and U.S. Pat. Nos. 5,292,658, 5,418,155, 5,683,888, 5,741,668, 5,777,079, 5,804,387, 5,874,304, 5,876,995, and 5,925,558.

In other embodiments, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; enzymes such as horseradish peroxidase, alkaline phosphatases, luciferases, etc; and cell surface markers, etc.

In a preferred embodiment, the secondary label is a binding partner pair. For example, the label may be a hapten or antigen, which will bind its binding partner. In a preferred embodiment, the binding partner can be attached to a solid support to allow separation of components containing the label and those that do not. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid—nucleic acid binding proteins pairs are also useful. In general, the smaller of the pair is attached to the system component for incorporation into the assay, although this is not required in all embodiments. Preferred binding partner pairs include, but are not limited to, biotin (or imino-biotin) and streptavidin, digeoxinin and Abs, etc.

In a preferred embodiment, the binding partner pair comprises a primary detection label (for example, attached to the assay component) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵-10⁻⁹ M⁻¹, being preferred and less than about 10⁻⁷-10⁻⁹ M⁻¹ being particularly preferred.

In a preferred embodiment, the secondary label is a chemically modifiable moiety. In this embodiment, labels comprising reactive functional groups are incorporated into the assay component. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

### H. Production of NAP Conjugates

In general, once the expression vectors of the invention are made, they can follow one of two fates, which are merely exemplary: they are introduced into cell-free translation systems, to create libraries of nucleic acid/protein (NAP) conjugates that are assayed in vitro, or, preferably they are introduced into host cells where the NAP conjugates are formed; the cells may be optionally lysed and assayed accordingly.

In a preferred embodiment, the expression vectors are made and introduced into cell-free systems for translation, followed by the attachment of the NAP enzyme to the EAS, forming a nucleic acid/protein (NAP) conjugate. By “nucleic acid/protein conjugate” or “NAP conjugate” herein is meant a covalent attachment between the NAP enzyme and the EAS, such that the expression vector comprising the EAS is covalently attached to the NAP enzyme. Suitable cell free translation systems are known in the art. Once made, the NAP conjugates are used in assays as outlined below.

In a preferred embodiment, the expression vectors of the invention are introduced into host cells as outlined herein. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ precipitation, liposome fusion, lipofectin®, electroporation, viral infection, gene guns, etc. The candidate nucleic acids may stably integrate into the genome of the host cell (for example, with retroviral introduction, outlined herein) or may exist either transiently or stably in the cytoplasm (i.e. through the use of traditional plasmids, utilizing standard regulatory sequences, selection markers, etc.). Suitable host cells are outlined above, with eucaryotic, mammalian and human cells all preferred.

Many previously described methods involve peptide library expression in bacterial cells. Yet, it is understood in the art that translational machinery such as codon preference, protein folding machinery, and post-translational modifications of, for example, mammalian peptides, are unachievable or altered in bacterial cells, if such modifications occur at all. Peptide library screening in bacterial cells often involves expression of short amino acid sequences, which can not imitate a protein in its natural configuration. Screening of these small, sub-part sequences cannot effectively determine the function of a native protein in that the requirements for, for instance, recognition of a small ligand for its receptor, are easily satisfied by small sequences without native conformation. The complexities of tertiary structure are not accounted for, thereby easing the requirements for binding.

One advantage of the present invention is the ability to express and screen unknown peptides in their native environment and in their native protein conformation. The covalent attachment of the fusion enzyme to its corresponding expression vector allows screening of peptides in organisms other than bacteria. Once introduced into a eukaryotic host cell, the nucleic acid molecule is transported into the nucleus where replication and transcription occurs. The transcription product is transferred to the cytoplasm for translation and post-translational modifications. However, the produced peptide and corresponding nucleic acid molecule must meet in order for attachment to occur, which is hindered by the compartmentalization of eukaryotic cells. NAM enzyme-EAS recognition can occur in four ways, which are merely exemplary and do not limit the present invention in any way. First, the host cells can be allowed to undergo one round of division, during which the nuclear envelope breaks down. Second, the host cells can be infected with viruses that perforate the nuclear envelope. Third, specific nuclear localization or transporting signals can be introduced into the fusion enzyme. Finally, host cell organelles can be disrupted using methods known in the art.

The end result of the above-described approaches is the transfer of the expression vector into the same environment as the fusion enzyme. The non-covalent interaction between a DNA binding protein and attachment site of previously described expression libraries would not survive the procedures required to allow linkage of the fusion protein to its expression vector in eukaryotic cells. Other DNA-protein linkages described in the art, such as those using the bacterial P2 A DNA binding peptide, require the binding peptide to remain in direct contact with its coding DNA in order for binding to occur, i.e., translation must occur proximal to the coding sequence (see, for example, Lindahl, Virology 42:522-533 (1970)). Such linkages are only achievable in prokaryotic systems and cannot be produced in eukaryotic cells.

Once the NAM enzyme expression vectors have been introduced into the host cells, the cells are optionally lysed. Cell lysis is accomplished by any suitable technique, such as any of a variety of techniques known in the art (see, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Most methods of cell lysis involve exposure to chemical, enzymatic, or mechanical stress. Although the attachment of the fusion enzyme to its coding nucleic acid molecule is a covalent linkage, and can therefore withstand more varied conditions than non-covalent bonds, care should be taken to ensure that the fusion enzyme-nucleic acid molecule complexes remain intact, i.e., the fusion enzyme remains associated with the expression vector.

In a preferred embodiment, the NAP conjugate may be purified or isolated after lysis of the cells. Ideally, the lysate containing the fusion protein-nucleic acid molecule complexes is separated from a majority of the resulting cellular debris in order to facilitate interaction with the target. For example, the NAP conjugate may be isolated or purified away from some or all of the proteins and compounds with which it is normally found after expression, and thus may be substantially pure. For example, an isolated NAP conjugate is unaccompanied by at least some of the material with which it is normally associated in its natural (unpurified) state, preferably constituting at least about 0.5%, more preferably at least about 5% by weight or more of the total protein in a given sample. A substantially pure protein comprises at least about 75% by weight or more of the total protein, with at least about 80% or more being preferred, and at least about 90% or more being particularly preferred.

NAP conjugates may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, gel filtration, and chromatofocusing. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the NAP conjugate. In some instances no purification will be necessary.

Thus, the invention provides for NAP conjugates that are either in solution, optionally purified or isolated, or contained within host cells. Once expressed and purified if necessary, the NAP conjugates are useful in a number of applications, including in vitro and ex vivo screening techniques. One of ordinary skill in the art will appreciate that both in vitro and ex vivo embodiments of the present inventive method have utility in a number of fields of study. For example, the present invention has utility in diagnostic assays and can be employed for research in numerous disciplines, including, but not limited to, clinical pharmacology, functional genomics, pharamcogenomics, agricultural chemicals, environmental safety assessment, chemical sensor, nutrient biology, cosmetic research, and enzymology.

The test compounds or “target molecules” or grammatical equivalents herein is meant a molecule for which an interaction is sought; this term will be generally understood by those in the art. Target molecules include both biological and non-biological targets. Biological targets refer to any defined and non-defined biological particles, such as macromolecular complexes, including viruses, cells, tissues and combinations, that are produced as a result of biological reactions in cells. Non-biological targets refer to molecules or structure that are made outside of cells as a result of either human or non-human activity. The inventive library can also be applied to both chemically defined targets and chemically non-defined targets. “Chemically defined targets” refer to those targets with known chemical nature and/or composition; “chemically non-defined targets” refer to targets that have either unknown or partially known chemical nature/composition.

Thus, suitable target molecules encompass a wide variety of different classes, including, but not limited to, cells, viruses, proteins (particularly including enzymes, cell-surface receptors, ion channels, and transcription factors, and proteins produced by disease-causing genes or expressed daring disease states), carbohydrates, fatty acids and lipids, nucleic acids, chemical moieties such as small molecules, agricultural chemicals, drugs, ions (particularly metal ions), polymers and other biomaterials. Thus for example, binding to polymers (both naturally occurring and synthetic), or other biomaterials, may be done using the methods and compositions of the invention.

### I. Isolation and Detection of NAP Conjugates Bound by Target Molecule

To isolate and detect a NAP conjugate that is bound by a target molecule, either the NAP conjugate or the target molecule can be non-diffusably bound to an insoluble support (e.g., a solid support) having isolated sample receiving areas (e.g. a microtiter plate, an array, etc.). The insoluble support may be made of any composition to which the assay component can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. Alternatively, bead-based assays may be used, particularly with use with fluorescence activated cell sorting (FACS). The particular manner of binding the assay component is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the use of labeled components (e.g. the assay component is biotinylated and the surface comprises strepavidin, etc.) the synthesis of the target on the surface, etc. Following binding of the NAP conjugate or target molecule, excess unbound material is removed by suitable methods including, for example, chemical, physical, and biological separation techniques. The sample receiving areas may then be blocked through incubation with bovine serum albumin (BSA), casein or other innocuous protein or other moiety.

In a preferred embodiment, the target molecule is bound to the support, and a NAP conjugate is added to the assay. Alternatively, the NAP conjugate is bound to the support and the target molecule is added. Novel binding agents include specific antibodies, non-natural binding agents identified in screens of chemical libraries, peptide analogs, etc. Of particular interest are screening assays for agents that have a low toxicity for human cells. Determination of the binding of the target and the candidate protein is done using a wide variety of assays, including, but not limited to labeled in vitro protein-protein binding assays, electrophoretic mobility shift assays, immunoassays for protein binding, the detection of labels, functional assays (phosphorylation assays, etc.) and the like.

The determination of the binding of the candidate protein to the target molecule may be done in a number of ways. In a preferred embodiment, one of the components, preferably the soluble one, is labeled, and binding determined directly by detection of the label. For example, this may be done by attaching the NAP conjugate to a solid support, adding a labeled target molecule (for example a target molecule comprising a fluorescent label), removing excess reagent, and determining whether the label is present on the solid support. This system may also be run in reverse, with the target (or a library of targets) being bound to the support and a NAP conjugate, preferably comprising a primary or secondary label, is added. For example, NAP conjugates comprising fusions with GFP or a variant may be particularly useful. Various blocking and washing steps may be utilized as is known in the art.

As will be appreciated by those in the art, it is also possible to contact the NAP conjugates and the targets prior to immobilization on a support.

In a preferred embodiment, the solid support is in an array format; that is, a biochip is used which comprises one or more libraries of either targets or NAP conjugates attached to the array. This can find particular use in assays for nucleic acid binding proteins, as nucleic acid biochips are well known in the art. In this embodiment, the nucleic acid targets are on the array and the NAP conjugates are added. Similarly, protein biochips of libraries of target proteins can be used, with labeled NAP conjugates added. Alternatively, the NAP conjugates can be attached to the chip, either through the nucleic acid or through the protein components of the system. This may also be done using bead based systems; for example, for the detection of nucleic acid binding proteins, standard “split and mix” techniques, or any standard oligonucleotide synthesis schemes, can be run using beads or other solid supports, such that libraries of sequences are made. The addition of NAP conjugate libraries then allows for the detection of candidate proteins that bind to specific sequences. In some embodiments, only one of the components is labeled; alternatively, more than one component may be labeled with different labels.

In a preferred embodiment, the binding of the candidate protein is determined through the use of competitive binding assays. In this embodiment, the competitor is a binding moiety known to bind to the target molecule such as an antibody, peptide, binding partner, ligand, etc. Under certain circumstances, there may be competitive binding as between the target and the binding moiety, with the binding moiety displacing the target.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound. Similarly, ELISA techniques are generally preferred.

A variety of other reagents may be included in the screening assays. These include reagents such as, but not limited to, salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, co-factors such as cAMP, ATP, etc., may be used. The mixture of components may be added in any order that provides for the requisite binding.

“Modulation” or “alteration” in this context includes an increase in activity, a decrease in activity, or a change in the type or kind of activity present. Thus, in this embodiment, the candidate protein should both bind to the target (although this may not be necessary), and alter its biological or biochemical activity as defined herein. The methods include both in vitro screening methods, as are generally outlined above, and ex vivo screening of cells for alterations in the presence, distribution, activity or amount of the target. Alternatively, a candidate peptide can be identified that does not interfere with target activity, which can be useful in determining drug-drug interactions.

Once a “hit” (protein ligand of the target molecule) is found, the NAP conjugate is retrieved to allow identification of the candidate protein. Retrieval of the NAP conjugate can be done in a wide variety of ways, as will be appreciated by those in the art and will also depend on the type and configuration of the system being used. In an embodiment, as outlined above, a rescue tag or “retrieval property” is used. A “retrieval property” is a property that enables isolation of the fusion enzyme when bound to the target. For example, the target can be constructed such that it is associated with biotin, which enables isolation of the target-bound fusion enzyme complexes using an affinity column coated with streptavidin. Alternatively, the target can be attached to magnetic beads, which can be collected and separated from non-binding candidate proteins by altering the surrounding magnetic field. Alternatively, when the target does not comprise a rescue tag, the NAP conjugate may comprise the rescue tag. For example, affinity tags may be incorporated into the fusion proteins themselves. Similarly, the fusion enzyme-nucleic acid molecule complex can be also recovered by immunoprecipitation. Alternatively, rescue tags may comprise unique vector sequences that can be used to PCR amplify the nucleic acid encoding the candidate protein. In the latter embodiment, it may not be necessary to break the covalent attachment of the nucleic acid and the protein, if PCR sequences outside of this region (that do not span this region) are used.

In a preferred embodiment, after isolation of the NAP conjugate of interest, the covalent linkage between the fusion enzyme and its coding nucleic acid molecule can be severed using, for instance, nuclease-free proteases, the addition of non-specific nucleic acid, or any other conditions that preferentially digest proteins and not nucleic acids.

The nucleic acid molecules are purified using any suitable methods, such as those methods known in the art, and are then available for further amplification, sequencing or evolution of the nucleic acid sequence encoding the desired candidate protein. Suitable amplification techniques include all forms of PCR, OLA, SDA, NASBA, TMA, Q-βR, etc. Subsequent use of the information of the “hit” is discussed below.

In a preferred embodiment, the NAP conjugates are used in ex vivo screening techniques. In this embodiment, the expression vectors of the invention are introduced into host cells to screen for candidate proteins with a desired property, e.g., capable of altering the phenotype of a cell. An advantage of the present inventive method is that screening of the fusion enzyme library can be accomplished intracellularly. One of ordinary skill in the art will appreciate the advantages of screening candidate proteins within their natural environment, as opposed to lysing the cell to screen in vitro. In ex vivo or in vivo screening methods, variant peptides are displayed in their native conformation and are screened in the presence of other possibly interfering or enhancing cellular agents. Accordingly, screening intracellularly provides a more accurate picture of the actual activity of the candidate protein and, therefore, is more predictive of the activity of the peptide ex vivo or in vivo. Moreover, the effect of the candidate protein on cellular physiology can be observed. Thus, the invention finds particular use in the screening of eucaryotic cells.

Ex vivo and/or in vivo screening can be done in several ways. In a preferred embodiment, the target need not be known; rather, cells containing the expression vectors of the invention are screened for changes in phenotype. Cells exhibiting an altered phenotype are isolated, and the target to which the NAP conjugate bound is identified as outlined below, although as will be appreciated by those in the art and outlined herein, it is also possible to bind the fusion polypeptide and the target prior to forming the NAP conjugate. Alternatively, the target may be added exogeneously to the cell and screening for binding and/or modulation of target activity is done. In the latter embodiment, the target should be able to penetrate the membrane, by, for instance, direct penetration or via membrane transporting proteins, or by fusions with transport moieties such as lipid moieties or HIV-tat, described below.

In general, experimental conditions allow for the formation of NAP conjugates within the cells prior to screening, although this is not required. That is, the attachment of the NAM fusion enzyme to the EAS may occur at any time during the screening, either before, during or after, as long as the conditions are such that the attachment occurs prior to mixing of cells or cell lysates containing different fusion nucleic acids.

As will be appreciated by those in the art, the type of cells used in this embodiment can vary widely. Basically, any eucaryotic or procaryotic cells can be used, with mammalian cells being preferred, especially mouse, rat, primate and human cells. The host cells can be singular cells, or can be present in a population of cells, such as in a cell culture, tissue, organ, organ system, or organism (e.g., an insect, plant or animal). As is more fully described below, a screen will be set up such that the cells exhibit a selectable phenotype in the presence of a candidate protein. As is more fully described below, cell types implicated in a wide variety of disease conditions are particularly useful, so long as a suitable screen may be designed to allow the selection of cells that exhibit an altered phenotype as a consequence of the presence of a candidate agent within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog.

In one embodiment, the cells may be genetically engineered, that is, contain exogeneous nucleic acid, for example, to contain target molecules. If necessary, the cells are treated to conditions suitable for the expression of the fusion nucleic acids (for example, when inducible promoters are used), to produce the candidate proteins.

Thus, the methods of the present invention preferably comprise introducing a molecular library of fusion nucleic acids or expression vectors into a plurality of cells, thereby creating a cellular library. Preferably, two or more of the nucleic acids comprises a different nucleotide sequence encoding a different candidate protein. The plurality of cells is then screened for a cell exhibiting an altered phenotype. The altered phenotype is due to the presence of a candidate protein.

By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptability, latency, adhesion, and uptake of viruses and bacterial pathogens; etc. By “capable of altering the phenotype” herein is meant that the candidate protein can change the phenotype of the cell in some detectable and/or measurable way.

The altered phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc.

In a preferred embodiment, once a cell with an altered phenotype is detected, the cell is isolated from the plurality which do not have altered phenotypes. This may be done in any number of ways, as is known in the art, and will in some instances depend on the assay or screen. Suitable isolation techniques include, but are not limited to, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes, etc.

In a preferred embodiment, as outlined above, the NAP conjugate is isolated from the positive cell. This may be done in a number of ways. In a preferred embodiment, primers complementary to DNA regions common to the NAP constructs, or to specific components of the library such as a rescue sequence, defined above, are used to “rescue” the unique candidate protein sequence. Alternatively, the candidate protein is isolated using a rescue sequence. Thus, for example, rescue sequences comprising epitope tags or purification sequences may be used to pull out the candidate protein, using immunoprecipitation or affinity columns. In some instances, as is outlined below, this may also pull out the primary target molecule, if there is a sufficiently strong binding interaction between the candidate protein and the target molecule. Alternatively, the peptide may be detected using mass spectroscopy. Once rescued, the sequence of the candidate protein and fusion nucleic acid can be determined. This information can then be used in a number of ways, i.e., genomic databases.

For in vitro, ex vivo, and in vivo screening methods, once the “hit” has been identified, the results are preferably verified. There are a variety of suitable methods that can be used. In a preferred embodiment, the candidate protein is resynthesized and reintroduced into the target cells to verify the binding between the candidate protein and the test compound. This may be done using recombinant methods, e.g., by transforming naive cells with the expression vector (or modified versions, e.g., with the candidate protein no longer part of a fusion), or alternatively using fusions to the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990).

In a preferred embodiment, the methods and compositions of the invention can be performed using a robotic system. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

A wide variety of automatic components can be used to perform the present inventive method or produce the present inventive compositions, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In a preferred embodiment, Interchangeable pipet heads (single or multi-channel ) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some preferred embodiments, the instrumentation will include a detector, which can be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which can be done using any number of known heating and cooling systems, including Peltier systems.

In a preferred embodiment, the robotic apparatus includes a central processing unit which communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

The above-described methods of screening a pool of fusion enzyme-nucleic acid molecule complexes for a nucleic acid encoding a desired candidate protein are merely based on the desired target property of the candidate protein. The sequence or structure of the candidate proteins does not need to be known. A significant advantage of the present invention is that no prior information about the candidate protein is needed during the screening, so long as the product of the identified coding nucleic acid sequence has biological activity, such as specific association with a targeted chemical or structural moiety. The identified nucleic acid molecule then can be used for understanding cellular processes as a result of the candidate protein's interaction with the target and, possibly, any subsequent therapeutic or toxic activity.

### V. EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### A. Example 1

This example demonstrates the binding of an expressed fusion protein to its coding nucleic acid molecule.

Plasmid pML2000, encoding a recombinant Rep78—coding DNA fusion fragment, was constructed using methods known in the art (see, for example, Sambrook et al., supra). The plasmid, pML200 contained the following features: a DNA replication origin functional in E. coli; an SV40 replication origin functional in mammalian cells; a constitutive promoter that is active in the host cells, specifically the CMV promoter; and one copy of the AAV serotype 2 inverted terminal repeat (ITR) sequence. The orientation of the ITR in reference to other components was not significant. The nucleic acid sequence that was the source of the AAV ITR had the sequence: 5′-AGGAACCCCTAGTGATGGAGTTGGCCACT CCCTCTCTGCGCGCTCGCTCGCTCACTGAGGCCGCCCGGGCAA AGCCCGGGCG - 3′ (SEQ ID NO:6). The duplex of the ITR sequence was previously shown to be sufficient for interaction with a variant of Rep68 (Chiorini et al., 1994, supra).

The resultant plasmid DNA was amplified in E. coli and purified using a DNA maxiprep kit (Promega Inc., WI). The purified DNA was transfected into tissue cultured HEK 293 cells (ATCC, MD) via calcium phosphate precipitation or electroporation techniques. At 48 hours post-transfection, the cells were harvested and lysed with 1% of Triton X-100 in standard phosphate buffered saline (PBS). After centrifugation at 5000×g for 30 minutes, the supernatant was used for subsequent biochemical characterization.

Expression of pML2000 in host cells allows for (i) expression of the modified Rep78 protein as a fusion protein with a referenced partner, and (ii) covalent attachment of the fusion protein to the attachment signal in a viral or plasmid vector. The expression of recombinant eREP was determined by immununoblot analyses using either anti-HA antibody or anti-REP antibody. The specific antibody binding was visualized by ECL chemiluminescence system (Amersham-Pharmacia Biotech, IN). Expression of functional Rep78 proteins was previously demonstrated in the mammalian cell culture system (Li et al., J. Virol., 71, 5236-5243 (1997)).

The ability to form DNA-eREP complexes was tested by the following experiments. Host cells were transfected with two plasmids, pML2000 and pML2000(ΔITR), individually and in combination. For each of the referenced transfections, a total of 10 μg DNA was added in order to achieve a similar level of eREP protein expression. At 48 hours after transfection, the cells were harvested and protein lysates were prepared. To test covalent binding between the expressed eREP and the plasmid DNA, the lysates were first boiled for 5 minutes and immediately chilled on ice. An aliquot of boiled lysate from each sample was mixed with anti-REP antibody followed by incubation with an excess amount of protein A agarose (Sigma, Mont.). After an extensive wash, the protein A agarose beads were transferred to PCR tubes. The presence of bound plasmid was tested by polymerase chain reaction to amplify the regions specific for either plasmid. The transfected plasmid pML2000 was precipitated by protein A agarose while the pML2000 (ΔITR) was not precipitated. The formed eREP-pML2000 complex was heat-resistant, consistent with the covalent bonding between eREP and the expression plasmid pML2000. Furthermore, the interaction is ITR sequence-specific similar to previous in vitro and in vivo data (Yang et al., J. Virol., 66, 6058-6069, (1992); Chiorini et al., J. Virol., 68, 797-804 (1994)).

This example demonstrates the construction of a vector suitable for use in the present inventive methods. The results demonstrate that enzyme-vector complexes are formed following expression of the Rep protein, and that binding of the Rep protein to its coding vector is covalent.

### B. Example 2

The following example demonstrates a method of identifying and isolating a nucleic acid molecule encoding a gene product comprising a target property using an affinity column.

To retrieve a protein with a desired property, a chemical moiety, for example, FK506 (CalBiochem Inc., CA) was purchased and chemically attached to biotin using a commercial chemical linkage reagent. After conjugation, the compound was purified via standard chromatographic techniques and confirmed by NMR. To immobilize the compound, immobilon-4 96-well plates first were coated with 10 μg/ml streptavidin (SA). Following the coating, the biotinylated-FK506 in PBS was added to saturate all binding sites. After removal of the excess biotinylated-FK506, the coated wells then were blocked with 1% BSA in PBS. After washing, the immobilized compound was ready for affinity selection.

A library of lysates comprising fusion enzyme-expression vector complexes were prepared by first transfecting approximately 10⁸ mammalian HEK cells with cDNA libraries prepared from mouse RNA using routine molecular biology techniques. At 48 hours post-transfection, the cells were harvested and collected by centrifugation. The cells were lysed in the presence of proteinase inhibitors by the lysis procedures described in Example 1. The clarification of total crude lysate was carried out by centrifugation at 5000×g for 30 minutes. The prepared cell lysates were either stored at −80° C. or immediately used with immobilon-4 wells coated with biotinylated-FK506. After incubation with the biotinylated-FK506, the lysate was removed from the immobilon-4 plates. The wells were then washed extensively with PBS using the 12 well Nunc hand-held washer (Corning, N.Y.). The bound fusion enzyme-expression vector complexes were released from the biotinylated-FK506 by incubation with 1% trypsin. The recovered DNA was extracted twice with Tris-buffered phenol and precipitated using a standard ethanol precipitation procedure in the presence of 1 μg of glycogen. The precipitated DNA was washed once with 70% ethanol and transformed into bacteria using electroporation. The isolated DNA can be further subjected to further rounds of affinity selection as desired.

This example demonstrates the isolation of a nucleic acid encoding a peptide comprising a desired property, the ability to bind FK506, using the methods of the present invention.

### C. Example 3

The following example demonstrates a method of characterizing the cDNA fragment inserted into the expression vector to form a fusion enzyme library.

cDNA encoding peptides with desired properties can be characterized by employing ELISA procedures using standard protocols and antibodies specific for the NAM enzyme, e.g., Rep78. Thus, if a cDNA clone encodes a peptide that interacts with FK506, it is expected that the cell lysate comprising the referenced plasmid DNA will be specific to FK506 coated wells, but not streptavidin (SA)-coated or other negative control coated wells. Similarly, one expects that a control plasmid does not result in lysates that induce any ELISA signal.

After two rounds of affinity panning, performed as described in Example 2, individual colonies of bacterial transformants were randomly selected. Overnight cultures from single colonies in 3 ml of LB ampicillin (100 μg/ml) were used to isolate DNA using a standard miniprep DNA kit (Promega, Wis.). Expression of the eREP-variant peptide fusion proteins was achieved by transient transfection into HEK 293 cells. At 48 hours posttransfection, cell lysates were prepared as described in Example 2. Clarified lysates were used immediately for ELISA or stored at −70° C. To prepare ELISA, 96-well plates were first coated with SA alone or SA+biotin-FK506. The wells were then blocked with 1% BSA in phosphate buffered saline (PBS) at pH 7.4. After precoating with SA, the wells were washed three times with PBS supplemented with 0.05% Tween-20 (PBT). To initiate binding of the fusion enzyme-expression vector complexes to the well surface, 100 μl of 1:10 diluted lysate was added to each well. After 60 minutes at 4° C., the plates were washed four times with PBT. The binding of the eREP DNA-binding portion peptide of the fusion enzyme was detected using rabbit anti-REP antibody. After 4 washes with PBT, the plate was developed by adding alkaline phosphatase-conjugated goat anti-rabbit antibody (GIBCO-BRL, MD) in PBS/0.1% BSA (100 μl per well for 1 hr at 25° C.) followed by a 6 to 100-min treatment with p-nitrophenyl phosphate (4 mg/ml) in 1 M diethanolamine hydrochloride, pH 9.8/0.24 mM MgCl₂ (200 μl per well). Binding was quantified by monitoring optical density (O.D.) at 405 nm on an E-max plate reader (Molecular Devices Inc., CA). The negative controls consisted of wells coated with control glutathione S-transferase (GST) fusion or as otherwise indicated. Control plasmids, e.g., plasmids not comprising the coding sequence for a FK506-binding peptide, did not induce a signal in the ELISA assay. Fusion enzymes comprising a peptide with the target property, FK506 binding, were identified via the ELISA assay. All experiments were repeated at least once with similar results.

This example demonstrates a method of using a fusion enzyme library to identify a peptide comprising a desired activity (i.e., binding to FK506) and to identify a nucleic acid encoding a target function by virtue of the fusion enzyme-expression vector linkage.

### D. Example 4

The following example demonstrates a method of using a fusion enzyme library to identify a DNA binding peptide, the nucleic acid molecule encoding the DNA binding peptide, and the nucleic acid sequence recognized by the DNA binding peptide.

A fusion enzyme library is constructed as described in Example 1. A population of random DNA sequences is generated to provide the DNA binding substrate for the DNA binding peptide encoded by the fusion enzyme library. DNA synthesis resin (bead) is used to make a lead oligonucleotide of 25 bases (cassette I) containing a Not I restriction enzyme site. After synthesis, the resin is divided into four aliquots and allowed to proceed to the next step of synthesis, wherein an A, T, G, or C is added (each aliquot has a different base type added). After one cycle, the resin is mixed and divided into four aliquots for the subsequent cycle, in which another A, T, G, or C is added individually to each aliquot. The referenced mixing and dividing steps are repeated twelve times to generate 12 mer random oligonucleotide cassettes (ROC). The resin is then mixed, and an additional 20 base cassette is added (cassette II). The split-mix synthesis procedures allow for the generation of random oligonucleotide DNA fragments wherein the resin mixture has “one sequence per bead.” In other words, onto each bead is attached many copies of a single oligonucleotide.

To obtain double stranded DNA binding substrate, the resultant resin mix is washed with a buffer for Klenow enzyme. The washed resins are mixed with synthetic oligonucleotides and an extension primer that is complementary to cassette II. The mixture is heated to 80° C., slowly cooled to 25° C., and chilled to 4° C., which allows the extension primer to hybridize to the template. The resultant mixture of resins is incubated in Klenow buffer under standard conditions in the presence of dNTPs, such that an extension reaction is carried out. The resultant resin with double stranded DNA is then washed with standard PBS buffer and stored at 4° C. in the presence of sodium azide.

To identify genes or coding sequences for DNA binding proteins, the resins with attached DNA fragments are incubated with the fusion enzyme library encoding putative DNA binding peptides at 4° C. for 12 hours. The bead-REP fusion enzyme complexes are marked with a primary antibody directed against REP. Following the incubation, the mixture is incubated with magnetic beads comprising pre-conjugated secondary antibody. After incubation, the bead-resin mixture is heated to denature the protein and disconnect the magnetic bead—oligonucleotide resin complexes. The magnetic beads are removed using standard procedures, thereby isolating the co-precipitated non-magnetic DNA-resin. This material can be used for PCR amplification and sequencing analyses either as a pool or via single bead analyses procedures. Optionally, the resultant mixture is pelleted by centrifugation at 5000×g for 10 minutes and washed extensively with PBS. The bound protein-cDNA complexes on the resin are treated with proteinase K. The nucleic acids coding for the desired fusion enzyme are recovered by standard DNA preparation procedures. If desired, the recovered plasmids are introduced into mammalian hosts and used for the subsequent round(s) of affinity selection. The binding sequences recognized by the DNA binding peptide can be determined by sequencing PCR products of bound DNA to a particular NAM enzyme-DNA binding peptide fusion. The DNA binding peptide can be identified using protein analysis methods known in the art.

Collectively, the methods used herein allow for the generation of a series of cDNAs encoding DNA binding proteins and their corresponding binding sequences. For example, once a binding sequence has been identified using random oligonucleotides, a homology search can be carried out to determine all candidate sites in the human genome that represent possible binding sites for a given DNA binding protein. Conceivably, an integrated protein-DNA interaction map/database for the human genome then can be generated.

### E. Example 5

The following example demonstrates a method of identifying tissue-specific toxicity of estradiol (or an estrogen-related compound).

Estrogen is known to bind to estrogen receptors α and β. However, clinical and environmental studies of estrogen and estrogen-like compounds suggest that in addition to these two nuclear receptors, estrogen may also act on an array of other proteins that induce nontranscription-related effects. It has also been suggested that the side effects of and drug resistance to estrogen (and closely related compounds such as tamoxifen in cancer treatment) may involve nonnuclear receptors. See, e.g,. Dhingra, Cancer Invest 19:649-659 (2001).

There are a group of estrogen-like compounds that exist in our living environments. They are called environmental estrogens and are defined as xenobiotics that resemble estrogen structurally. These estrogens are divided into two broad categories: xeno-estrogens and phyto-estrogens. Environmental estrogens is thought to contribute to the increased incidence of reproductive disorders in the modem environment. See, e.g., Dubeyl et al., Hum Reprod Update 6:351-63 (2000); and Guillette, Growth Horm IGF Res 10 Suppl B, S45-50 (2000).

### 1. cDNA Library Construction in the REP-Mediated Protein Display Vector

The following describes exemplary procedures for making a REP-mediated protein display cDNA library suitable for toxicity profiling.

Fetal brain can be used as the RNA source for the cDNA library construction because (1) this organ is less differentiated compared to other tissues, and hence the resultant cDNA library will be more diverse; and (2) it has been shown that an estrogen receptor is expressed in this tissue. Simpkins et al., Am J Med 103:19S-25S (1997).

The vector carrying the library may contain a Rep78 gene and a recognition site (EAS) of Rep78. The Rep78-cDNAs fusion genes can be cloned into an expression cassette controlled by the CMV promoter and the poly-adenylation signal of the bovine growth hormone gene. A linker encoding (GGGGSGGGGS) (SEQ ID NO:7) is optionally placed between the Rep78 gene and the cDNA in the vector. The linker is used to facilitate independent folding of the protein modules, as used in, for example, single chain antibody engineering.

Total RNA of fetal brain is acquired from a commercial vendor. The MRNA is purified at least twice using the oligo-d(T) conjugated beads, and subjected to quality control by denaturing electrophoresis. A total of 1-2 μg of mRNA is used for cDNA synthesis. The SUPERSCRIPT II kit (LifeTechnology™) is used to produce the double-stranded cDNAs. A random hexamer primer, with an XbaI site in the 5′ end, is used for reverse transcription to synthesize the first strand of cDNA. The XbaI site in the primer will ensure that the subsequent cDNA cloning is directional. The oligo-d(T) primer can also be used as it tends to produce more full-length cDNA clones, although it may be biased toward the 3′ portion of the mRNA. The two libraries (made with and without oligo-d(T), respectively) may be used individually or in combination in affinity panning.

The second strand of the cDNA is labeled with a trace amount of ³²P during synthesis to quantify the cDNA yield and to indicate the cDNA size during size fractionation. The double-stranded cDNA is ligated to a NotI adaptor before being cloned into the NotI/XbaI sites of the vector. The resultant primary library may be stored in sub-library aliquots. The expected library complexity is in the range of 5×10⁶.

### 2. Synthesis of Biotinylated Estradiol

The following describes exemplary procedures for making biotinylated-estrogen suitable for affinity selection.

When biotinylating estradiol for isolating potential estradiol-binding proteins, two factors are taken into account: the derivatization site and the composition of the linker that connects the biotin and estradiol moieties. The derivatization site must be chosen to minimize counterproductive interactions between the introduced linker and the potential binding proteins. Earlier studies demonstrated that the ER (estrogen receptor) can tolerate the introduction of bulky derivatives in the B ring of the steroid at the C-7 position. Anstead et al., Steroids 62:268-303 (1997). Thus this site can be used for derivatization.

Linker composition is considered in two aspects: the hydrophobicity/hydrophilicity of the linker and the length of the linker. Since panning is conducted in an aqueous environment, a hydrophilic linker is preferably employed. The hydrophilicity can minimize hydrophobic interactions and prevent the linker from folding upon itself. Further, the linker must be long enough to allow simultaneous binding of the avidin beads to the biotin portion of the conjugate and any binding of a protein to the estradiol portion of the conjugate.

In accordance with these considerations, estradiol derivative 1 (made in six steps from estradiol; Skaddan et al., J Org Chem 64:8108-8121 (1999)) is ozonized and then decomposed using hydrogen peroxide and the bis-TBS protecting groups cleaved by tetrabutylammonium fluoride (TBAF) to yield carboxylic acid 2 (scheme 1; infra). The standard peptide coupling conditions of EDC/HOBt (Sheehan et al., J Am Chem Soc 95:875-879 (1973)) followed by treatment of the resulting activated ester with 3,3′-diamino-N-methyldipropyl amine delivers the immediate precursor to our estradiol-biotin conjugate 3. Finally, biotinylation is accomplished through the treatment of intermediate 3 with EZlirik-NHS-PEO4-Biotin (available from Pierce) to yield the desired estradiol-biotin conjugate 4.

Incorporation of 3,3′-diamino-N- methyldipropyl amine is important because it allows additional spacing between the molecule that we wish to display (estradiol) and thus facilitates the affinity isolation of all potential protein binding

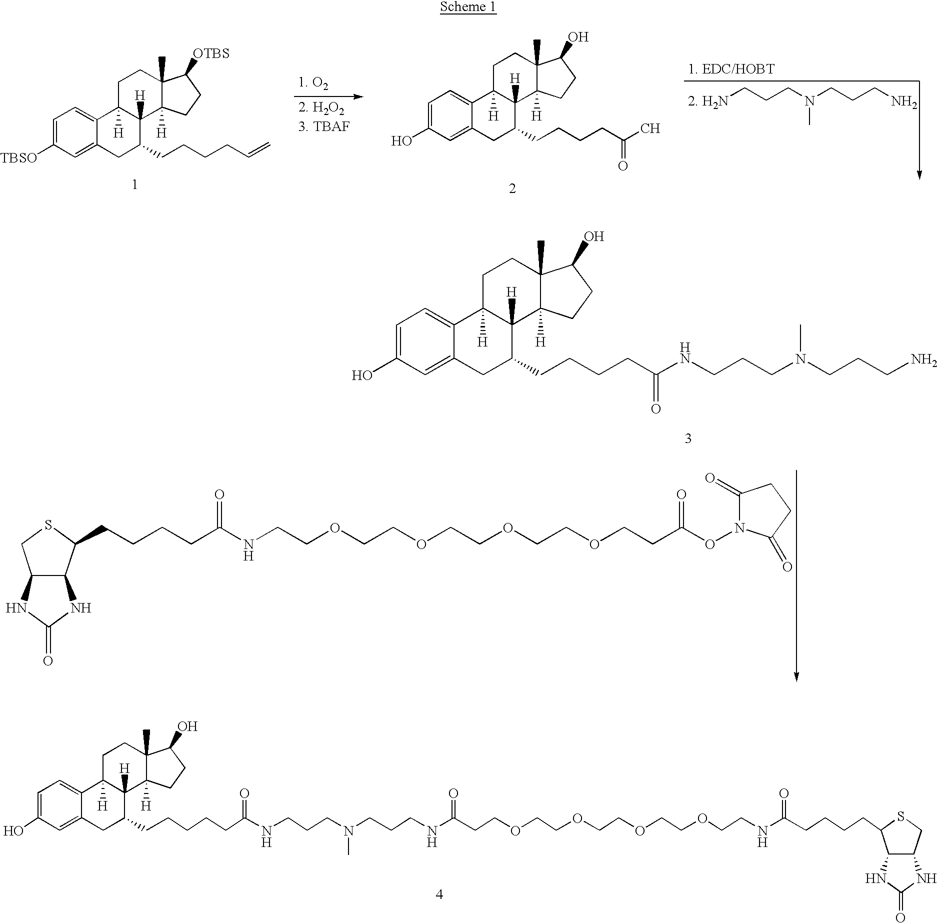

partners through an avidin column. This spacer will also promote water solubility by the incorporation of a charge group through protonation of the tertiary amine when prepared as its acid salt (trifluoroacetic acid/HCl/etc.). Use of the PEO4 linker is also preferred because it allows additional distance to be placed between the estradiol moiety and the biotin moiety while retaining the hydrophilic properties associated with PEG derivatives.

### 3. Affinity Screening

The following describes the procedures for affinity screening and isolation of candidate proteins that interact with biotinylated-estradiol.

The inserted figure highlights the steps in affinity selection. Typically, at least two (e.g., three) rounds of affinity selection are performed. Primary hits (NAP conjugates bound to estradiol) are confirmed by a competition assay before they are subjected to DNA sequencing and the results can be analyzed using nucleic acid sequence database. Four aspects critical for successful panning should be considered: (1) the multiplicity of the library, (2) the effective linkage between genotype and phenotype, (3) proper conditions of target-binder interaction and (4) the retrieval of complex and corresponding genetic materials.

A moderate to large amount of DNA is preferred during the DNA transfection to reach a high multiplicity. However, the amount of DNA should also be determined to achieve an appropriate balance to maintain a higher ratio of the corresponding protein-DNA linkage. The condition for target-binder interaction and retrieval of the estradiol-NAP complex are the most critical steps of the whole panning procedure. These stages can be affected by a variety of factors including the binding affinity, the abundance of the candidate NAP conjugate, and the potential activity of the candidate protein in the host cells. In a typical affinity selection, the labeled test compound (biotinylated estradiol in this case) is in excess, e.g., at least about 10- to 100-fold higher in concentration than the Kd of the interaction. An even higher concentration may be required to retrieve weaker binders that are also biologically relevant.

In an affinity screen where a biotinylated test compound is used, the possible background comes from non-specific interactions among the test compound, Rep78, the DNA expression vector, the solid surface, and nonspecific biotin, streptavidin and surface binders. Precaution should be exercised to minimize these possible backgrounds.

The genetic material in the NAP conjugate bound by the test compound can be recovered by transformation or by PCR. The most effective electroporation transformation has an efficiency of 10¹⁰ cfu/μg of DNA. Depending on the vector size, such transformation gives rise to a sensitivity threshold of approximately one colony per 1,000 copies. Transformation has a number of advantages, with no subcloning step needed and little bias to the gene. PCR recovery can also be used because it is a more sensitive method.

For a typical REP-mediated protein display screening, the first step is to introduce the sub-libraries into the host cells, e.g., human 293H cells. A time course is performed to determine the expression level of the fusion gene and the proper time for post-transfection cell harvesting. After being transfected for a certain period of time, the cells are washed by PBS, collected and weighed to determine the cell volume. The cells are then lysed under mild conditions in an equal volume of a lysis buffer containing 2% Triton X-100. The supernatant of the lysate will contain the functional library with NAP conjugates.

Streptavidin beads are then added into the lysate supernatant to remove any background streptavidin or solid surface binders. The biotinylated estradiol is then mixed with the pre-cleared lysate. After incubation at 4° C. the material is selectively precipitated by streptavidin beads that have been pre-blocked by the lysate from nontransfected cells. This step will enrich the biotin-estradiol-NAP complex.

The bound NAP conjugate may be released by SDS/proteinase K digestion. Alternatively, the NAP conjugate can be released by ligand competition with a high concentration of free estradiol, which may be more specific than the SDS/proteinase K elution. However, under some circumstances, this elution method may not be able to elute high affinity binders readily. The two elution methods may be combined in the screen. The DNA can then be recovered by PCR or transformation, and used for the next round of panning.

After two or three rounds of panning, the clones of the selected pools can be subjected to high throughput screening. The primary screening can be a single concentration determination. The screening assay can be an ELISA-type assay, a radioimmuno assay, a cell-based radioactive retention assay, or a homogenous assay. These assays are well established and can be readily applied to high throughput screening.

The cell-based radioactive retention assay may be preferred under some circumstances because it is physiologically more relevant. In this assay, DNAs from individual clones are transfected into 293 cells in a 96-well format. Twenty-four hours later, ³H-labeled estradiol is added into the cell culture. The ³H-labeled estradiol will diffuse into the cells in about 2-3 hrs. The cells are then washed extensively with PBS. Positive clones that have bound to estradiol can be identified by the retention of the radioactive signal in the cell. Clones obtained from the primary screening can be further screened by a competition assay with non-radiolabeled estradiol as well as counter-screened with non-specific inhibitors. An IC50 can be determined by the same assay.

If a screening assay requires the use of biotinylated compounds, one will confirm that the NAP conjugates recognize estradiol other than biotin, streptavidin, or the solid surface. This can be achieved by a competition assay using non-biotinylated estradiol as the inhibitor. Streptavidin and/or another compound can be used as a counter-screen to ensure the competition result.

In sum, the above-described procedures achieve the following objectives: (1) construction of a cDNA library suitable for the toxicity profiling based affinity screening; (2) production of a biotinylated, functional estradiol suitable for creating a protein-binding profile of estradiol; and (3) creation of protein-binding profiles that comprise lists of candidate proteins that interact with biotinylated estradiol.

Additional references that a skilled person may wish to consult for practicing this invention include: Boder et al., Nat Biotechnol 15:553-7 (1997); Caponigro et al., Proc Natl Acad Sci USA 95:7508-13 (1998); Carreras et al., Anal Biochem 298:57-61 (2001); Chiorini et al., J Virol 68:7448-57 (1994); Clackson et al., Trends Biotechnol 12:173-84 (1994); Colas et al., Nature 380:548-50 (1996); Crameri et al., Gene 137:69-75 (1993); Cull et al., Proc Natl Acad Sci USA 89:1865-9 (1992); Cunningham et al., Ann N Y Acad Sci 919:52-67 (2000); Davis et al., J Virol 74:2936-42 (2000); Doyle et al., Cell 85:1067-1076 (1996); Dunn, J Mol Biol 248:497-506 (1995); Efimov et al., Virus Genes 10:173-7 (1995); Jespers et al., Biotechnology (NY) 13, 378-82 (1995); Kay, Biochem J 314:361-85 (1996); Kim et al., Nature 378: 85-88 (1995); Kornau et al, Science 269:1737-1740 (1995); Li, Nat Biotechnol 18: 1251-6 (2000); Little et al., J Biotechnol 41:187-95 (1995); Lu et al, Biotechnology (NY) 13:366-72 (1995); Maruyama et al., Proc Natl Acad Sci USA 91:8273-7 (1994); Mattheakis et al., Proc Natl Acad Sci USA 91:9022-6 (1994); Matthews et al., Drug Discov Today 6:141-149 (2001); Mayer et al., Science 286:971-4 (1999); Norman et al., Science 285:591-5 (1999); Ren et al., Protein Sci 5:1833-43 (1996); Roberts et al., Proc Natl Acad Sci USA 94:12297-302 (1997); Rosenberg et al., inNovations 6:1-6 (1996); Santini et al., J Mol Biol 282, 125-35 (1998); Sche et al., Chem Biol 6:707-16 (1999); Schreiber, Science 251:283-7 (1991); Seed et al., Proc Natl Acad Sci USA 84: 3365-9 (1987); Smith, Science 228:1315-7 (1985); Songyang et al., Science 275:73-77 (1997); Sparks et al., J Biol Chem 269:23853-6 (1994); Sternberg et al., Proc Natl Acad Sci USA 92:1609-13 (1995); Walker et al., J Virol 71:2722-30 (1997); and Zweiger, Trends Biotechnol 17:429-36 (1999).

### Other Embodiments

While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the following claims.

What is claimed is:

1. A method of creating a protein-binding profile of a test compound, comprising:
contacting the test compound with a library of nucleic acid/protein (NAP) conjugates, wherein each of said NAP conjugates comprises:
(a) a fusion protein comprising
(i) a nucleic acid modifying (NAM) enzyme, and
(ii) a candidate protein; and
(b) an expression vector comprising
(i) a fusion nucleic acid comprising a coding sequence for said NAM enzyme and a coding sequence for said candidate protein, and
(ii) an enzyme attachment sequence (EAS);
wherein said EAS and said NAM enzyme are covalently linked, and
wherein at least two of said NAP conjugates comprise different candidate proteins;
detecting the binding of said test compound to a NAP conjugate in the library; and
identifying the candidate protein in the bound NAP conjugate by determining the nucleotide composition of the coding sequence for the candidate protein in said bound NAP conjugate,
thereby creating a protein-binding profile of said test compound comprising a list of candidate proteins to which said test compound binds.

2. The method of claim 1, wherein said NAM enzyme is a Rep protein.

3. The method of claim 1, wherein each of said NAP conjugates are produced by eukaryotic host cells containing the expression vectors.

4. The method of claim 1, wherein the coding sequence for the candidate protein is derived from a cDNA library.

5. The method of claim 1, further comprising the step of determining the binding affinity between the test compound and the bound NAP conjugate.

6. A method of determining the toxicity of a compound, comprising:
providing
(a) a first protein-binding profile of a first compound, said first compound being known to have a toxic effect in an animal species, and
(b) a second protein-binding profile of a second compound, said first and second profiles each being obtained by a method of claim 1, wherein the coding sequence for the candidate protein is derived from said animal species; and
comparing said first and second profiles;
wherein substantial similarity between the two profiles is indicative of said second compound's having said toxic effect in said animal species.

7. A method of determining the toxicity of a compound, comprising:
providing
(a) a first protein-binding profile of said compound, said compound being known to have a toxic effect in a first animal species, and said first profile being obtained by a method of claim 1 in which the coding sequence for the candidate protein is derived from said first animal species; and
(b) a second protein-binding profile of said compound, said second profile being obtained by a method of claim 1 in which the coding sequence for the candidate protein is derived from a second animal species;
comparing said first and second profiles;
wherein substantial similarity between the two profiles is indicative of said compound's having said toxic effect in said second animal species.

8. A method of determining the toxicity of a compound, comprising:
providing
(a) a first protein-binding profile of said compound, said compound being known to have a toxic effect at a first organ of an animal species, and said first profile being obtained by a method of claim 1 in which the coding sequence for the candidate protein is derived from said first organ; and
(b) a second protein-binding profile of said compound, said second profile being obtained by a method of claim 1 in which the coding sequence for the candidate protein is derived from a second organ of said animal species;
comparing said first and second profiles;
wherein substantial similarity between the two profiles is indicative of said compound's having said toxic effect in said second organ.

9. A method of determining the toxicity of a compound, comprising:
providing
(a) a first protein-binding profile of said compound, said compound being known to have a toxic effect in an animal species at a first developmental stage, and said first profile being obtained by a method of claim 1 in which the coding sequence for the candidate protein is derived from said first animal species at said first developmental stage; and
(b) a second protein-binding profile of said compound, said second profile being obtained by a method of claim 1 in which the coding sequence for the candidate protein is derived from said animal species at a second developmental stage;
comparing said first and second profiles;
wherein substantial similarity between the two profiles is indicative of said compound's having said toxic effect in said second animal species at said second developmental stage.

10. A method of determining the toxicity of a compound, comprising:
providing a protein-binding profile of the compound, said profile being obtained by a method of claim 1,
determining whether the protein-binding profile includes one or more of the following: liver enzymes; cytochrome proteins; proteins encoded by multiple drug resistance genes; p450; and proteins associated with glutathione regulation, DNA repair, transcription regulation, structural maintenance, cell cycle control, and/or apoptosis; heat shock proteins; and housekeeping genes;
whereby such inclusion is indicative of said compound's having a toxic effect.
